(19) United States
(12) Patent Application Publication
(10) Pub. No.: US 2024/0335540 A1
(43) Pub. Date: Oct. 10, 2024

(54) COMPOSITIONS AND METHODS FOR TARGETED DELIVERY OF THERAPEUTIC AND/OR DIAGNOSTIC SPECIES

(71) Applicant: The Penn State Research Foundation, University Park, PA (US)

(72) Inventors: Daniel J. Hayes, State College, PA (US); Mohammad Abu-Laban, State College, PA (US); Nicholas Andrew ALDEN, State College, PA (US); Julien Henri Arrizabalaga, State College, PA (US)

(21) Appl. No.: 18/744,417

(22) Filed: Jun. 14, 2024

Related U.S. Application Data
(63) Continuation of application No. PCT/US2022/053146, filed on Dec. 16, 2022.
(60) Provisional application No. 63/291,133, filed on Dec. 17, 2021.

(51) Int. Cl. A61K 41/00; A61K 9/51; A61K 47/69; A61P 35/00; B82Y 5/00; C12N 15/113

(52) U.S. Cl. CPC A61K 41/0042; A61K 9/5115; A61K 47/6929; A61P 35/00; C12N 15/113; B82Y 5/00; C12N 2310/141

(57) ABSTRACT

In one aspect, compositions are described herein. A composition described herein comprises a nanoparticle, a therapeutic species, and a linker joining the nanoparticle to the therapeutic species. The linker joining the nanoparticle to the therapeutic species comprises a Diels-Alder cyclo-addition reaction product. Additionally, in some embodiments, the nanoparticle is a core-shell-shell metal nanoparticle.

### CROSS REFERENCE TO RELATED APPLICATIONS

This application is a continuation of International Application No. PCT/US2022/053146, filed Dec. 16, 2022, which claims priority pursuant to 35 U.S.C. § 119 to United States Provisional Patent Application No. 63/291,133, filed on Dec. 17, 2021, the entirety of which is hereby incorporated herein by reference in its entirety.

### STATEMENT REGARDING FEDERALLY SPONSORED RESEARCH

This invention was made with government support under Grant No. DE024790 awarded by the National Institutes of Health, under Grant No. CBET1722533 awarded by the National Science Foundation and under Grant No. W81XWH-18-1-0115 awarded by the U.S. Army/MRMC. The Government has certain rights in the invention.

### FIELD

The invention is generally related to delivery of therapeutic and/or diagnostic species to a biological compartment, and, more specifically, to use of nanoparticles with a therapeutic agent attached with a cleavable linker.

### BACKGROUND

Techniques for the stimuli-responsive delivery of therapeutic and/or diagnostic species have been of increasing interest in recent years. Several techniques have been explored, including for drug delivery. However, realizing control of drug delivery (e.g., temporal control, locational control, and/or control of the amount of drug delivered to a biological compartment) remains a significant challenge. Therefore, there is a need for improved compositions and methods for targeted delivery of therapeutic and/or diagnostic species.

### SUMMARY

Compositions and methods are described herein that, in some embodiments, overcome one or more of the aforementioned deficiencies and/or concerns of prior stimuli-responsive delivery techniques. For example, in some cases, compositions and methods described herein provide improved locational control of therapeutic and/or diagnostic species delivery. In one aspect, compositions are described herein. In some instances, a composition described herein comprises a nanoparticle, a therapeutic species, and a linker joining the nanoparticle to the therapeutic species. The linker joining the nanoparticle to the therapeutic species comprises a Diels-Alder cyclo-addition reaction product.

The nanoparticle of a composition described herein can be any nanoparticle not inconsistent with the objectives of this disclosure. In some cases, for example, the nanoparticle is a metal nanoparticle. The metal nanoparticle can be formed from silver or gold, for instance. Moreover, in some embodiments, a metal nanoparticle also can have a plasmon resonant or resonance frequency in the visible or near infrared region of the electromagnetic spectrum. In other cases, the nanoparticle of a composition described herein is a magnetic nanoparticle. More particularly, in some embodiments, the magnetic nanoparticle can be formed from a magnetic metal or metal oxide. For example, a nanoparticle described herein can be formed from Fe₂O₃, Fe₃O₄, MnFe₂O₄, CoFe₂O₄, or NiFe₂O₄. Further, in some embodiments, the magnetic nanoparticle can exhibit a specific loss power (SLP) greater than 20 W/g at a frequency of 850 kHz and/or a specific loss power (SLP) greater than 2 W/g at a frequency of 200 kHz.

In some embodiments, a nanoparticle described herein comprises a core-shell-shell nanoparticle. Such a core-shell-shell (CSS) nanoparticle can comprise a core, a first shell overlying the core, and a second shell overlying the first shell. As understood by a person of ordinary skill in the art, the first shell can fully or substantially fully (e.g., at least 95%, at least 98% or at least 99%) cover or “overcoat” the surface of the core, such that the core is contained within or “wrapped” by the first shell. Similarly, the second shell can fully or substantially fully cover or “overcoat” the surface of the first shell, such that the core and first shell are both contained within or “wrapped” by the second shell. The core, first shell, and second shell can be formed from any material not inconsistent with the objectives of the present disclosure. For example, in some cases, the core, first shell, and/or second shell comprise or are formed from a metal or a combination, alloy, or mixture of metals, where the “metals” are elemental or zero oxidation state metals, as opposed to metal ions such as may be found in a metal oxide. The metal or combination, alloy, or mixture of metals can also be the same or different for the core and one or more shells. For example, in some cases, the nanoparticle comprises a gold-silver-gold core-shell nanoparticle. Other nanoparticle structures are also possible, as described further hereinbelow.

Additionally, in some instances, a nanoparticle described herein has an optical absorbance peak in the near infrared (NIR) region of the electromagnetic spectrum, such as between 800 nm and 900 nm.

The therapeutic species of a composition described herein can be any therapeutic species not inconsistent with the objectives of this disclosure. In some embodiments, for example, the therapeutic species is a small molecule, a nucleic acid, a peptide, a protein, or any combination thereof. The nucleic acid can comprise a plasmid, a small interfering RNA (“siRNA”), a micro-RNA (“miRNA”), an miRNA mimic, or any combination thereof. Further, the therapeutic species can be covalently bound to the linker, and the linker can be covalently bound to the nanoparticle.

The linker of a composition described herein comprises or includes a Diels-Alder cycloaddition reaction product. Any such reaction product can be included in, or form at least part of, a linker described herein. For instance, in some embodiments, the Diels-Alder cycloaddition reaction product is a reaction product of a dienophile and a furan, a reaction product of a dienophile and a thiophene, or a reaction product of a dienophile and a pyrrole. Moreover, in some such cases, the Diels-Alder cyclo-addition reaction product has a retro-Diels-Alder activation temperature between 45° C. and 180° C., between 45° C. and 170° C., between 45° C. and 160° C., between 45° C. and 150° C., between 45° C. and 120° C., or between 45° C. and 80° C.

In another aspect, methods of delivering a therapeutic species to a biological compartment are described herein. In some instances, such a method comprises two steps. The first step is disposing a composition described herein in the biological compartment. Any composition described herein can be used. For example, in some cases, the composition comprises a metal or magnetic nanoparticle and a linker joining the nanoparticle to the therapeutic species, wherein the linker comprises a Diels-Alder cyclo-addition product. Turning again to exemplary methods described herein, the second step of a method can comprise initiating a retro Diels-Alder reaction to decompose the Diels-Alder cyclo-addition product of the composition. In this manner, the linker can be severed and the therapeutic species can be decoupled from the nanoparticle, including in a desired location.

Further, the step of initiating the retro Diels-Alder reaction can comprise heating the nanoparticle (e.g., a metal or magnetic nanoparticle) to an activation temperature of the retro Diels-Alder reaction, which can be between 45° C. and 160° C. In some such embodiments, the nanoparticle (e.g., a metal nanoparticle) has a plasmon resonant frequency, and heating the nanoparticle to the activation temperature comprises exposing the nanoparticle to electromagnetic radiation comprising a frequency corresponding to the plasmon resonant frequency. In other cases, the nanoparticle is a magnetic nanoparticle and heating the magnetic nanoparticle to the activation temperature comprises exposing the magnetic nanoparticle to an alternating magnetic field.

In another aspect, methods of inducing tissue regeneration are described herein. In some instances, the method comprises disposing a composition described herein in a biological compartment. In some cases, the composition comprises a metal or magnetic nanoparticle and a linker joining the nanoparticle to a therapeutic species, wherein the linker comprises a Diels-Alder cycloaddition product. Any Diels-Alder cycloaddition product described herein can be used. In some embodiments, the therapeutic species can comprise a tissue regenerative species, such as an osteogenic, chondrogenic, endotheliologenic, or myogenic modulators.

The method of inducing tissue regeneration can further comprise initiating a retro Diels-Alder reaction to decompose the Diels-Alder cycloaddition product of the composition and releasing the therapeutic species into the biological compartment. In some embodiments, the released therapeutic species induces osteogenic upregulation.

The step of initiating the retro Diels-Alder reaction can comprise heating the nanoparticle (e.g., a metal or magnetic nanoparticle) to an activation temperature of the retro Diels-Alder reaction, as previously described herein.

In another aspect, methods of treating cancer are described herein. In some instances, the method comprises disposing a composition described herein in a biological compartment. In some cases, the composition comprises a metal or magnetic nanoparticle and a linker joining the nanoparticle to a therapeutic species, wherein the linker comprises a Diels-Alder cycloaddition product. Any Diels-Alder cycloaddition product described herein can be used. In some embodiments, the therapeutic species can comprise an anti-cancer agent. Exemplary embodiments of anti-cancer agent can comprise Paclitaxil, Afatinib, Dimaleate, Bortezomib, Carfilzomib, Doxorubicin, Fluorouracil, miRNA 148b, -135, -124, -101, -29c, -15a, and -34 (MRX34) mimics.

Further non-limiting examples of therapeutic micro-RNA include miRNA-34a-5p, miRNA-7-5p, miRNA-218-5p, miRNA-148b-3p, miRNA433-3p, miRNA-181a-5p, or a combination of two or more of the foregoing. In some embodiments, these miRNA may demonstrate advantages in treating certain types of cancer and/or tumors such as cancers and/or tumors of nervous system tissue (e.g., glioblastoma multiform, astrocytoma, etc.).

The method of treating cancer can further comprise initiating a retro Diels-Alder reaction to decompose the Diels-Alder cycloaddition product of the composition and releasing the anti-cancer agent into the biological compartment. In some embodiments, the released therapeutic species induces cellular apoptosis, inhibits metastasis, suppresses tumor growth (“tumor suppressor”), or inhibits cancer stemness.

The step of initiating the retro Diels-Alder reaction can comprise heating the nanoparticle (e.g., a metal or magnetic nanoparticle) to an activation temperature of the retro Diels-Alder reaction, as previously described herein. These and other embodiments are described in more detail in the detailed description that follows.

### BRIEF DESCRIPTION OF THE DRAWINGS

The invention will now be described by way of example, with reference to the accompanying figures, of which:

FIG. 1 illustrates absorbance spectra for metal nanoparticles.

FIG. 2 illustrates a plot of specific loss power (“SLP”) values for magnetic nanoparticles.

FIG. 3 illustrates a plot of intrinsic loss power (“ILP”) values for magnetic nanoparticles.

FIG. 4 schematically illustrates a linker covalently bound to a therapeutic species.

FIG. 5 schematically illustrates a Diels-Alder reaction between a diene and a dienophile 6-maleimide hexanoic acid to form a linker.

FIG. 6 schematically illustrates an exemplary retro-Diels-Alder reaction of the product of FIG. 5.

FIG. 7 graphically illustrates cleavage temperatures of different linkers in a retro-Diels-Alder reaction.

FIG. 8 displays the calculated forward and reverse ΔHᵣₓₙ for formation of three different linkers.

FIG. 9 shows fluorescence intensity of a control (non-treated) sample and a chemically cleaved (treated) sample including a product formed by reacting the furan-based linker with EDC/NHS and a fluorescently-labeled si-RNA.

FIG. 10 graphically illustrates a temperature dependency of a retro Diels-Alder reaction (and release of a fluorescent si-RNA) for a furan-based linker product (connected to a nanoparticle) after 1 hour of heating at 37° C., 60° C., and 80° C.

FIG. 11 graphically illustrates a temperature dependency of a retro Diels-Alder reaction (and release of the fluorescent si-RNA) for a thiophene-based linker product (connected to a nanoparticle) after 2 hours of heating at 37° C., 60° C., and 80° C.

FIG. 12 illustrates transmission electron microscope (TEM) images of MnFe₂O₄ and Fe₃O₄ nanoparticles.

FIG. 13 illustrates TEM images of CoFe₂O₄ nanoparticles.

FIG. 14 illustrates TEM images of NiFe₂O₄ Nanoparticles.

FIG. 15 illustrates high resolution TEM (HRTEM) images of CoFe₂O₄ and NiFe₂O₄ nanoparticles.

FIG. 16 graphically illustrates Hysteresis Loops of CoFe₂O₄, MnFe₂O₄ and Fe₃O₄ nanoparticles.

FIG. 17 graphically illustrates a magnetic field sweep.

FIG. 18 graphically illustrates second harmonic intensity of a thiophene, furan, or pyrrole-based linker connecting an si-RNA to a plasmonic silver nanoparticle (SNP).

FIG. 19 graphically illustrates second harmonic intensity at particular temperatures as a function of time for a composition comprising a siRNA-SNP with a pyrrole-based linker.

FIG. 20 graphically illustrates second harmonic intensity at particular temperatures as a function of time for a composition comprising a plasmonic silver nanoparticle connected to si-RNA via a furan-based linker.

FIG. 21 graphically compares decay constants for a composition with a furan-based linker (FFT) to a composition with a thiophene-based linker (TMT).

FIG. 22 graphically illustrates relative release of the fluorescently labeled miRNA from the heterodimeric Fe₃O₄/Au was measured with respect to time at 155 kHz and 9.76 mT.

FIG. 23A is a negative control showing fluorescence of the PAM212 cells in the presence of silver nanoparticles.

FIG. 23B is a positive control showing PAM212 cells transfected with FAM-tagged miRN-148b.

FIG. 23C shows PAM212 cells in the presence of a furan-based linker conjugated with a (fluorescein) FAM-tagged miRN-148b and an SNP, where the composition has not been light activated.

FIG. 23D shows PAM212 cells in the presence of a furan-based linker conjugated with a FAM-tagged miRN-148b and an SNP composition, where the composition has been light activated.

FIG. 24A is a negative control showing fluorescence of Ras-induced keratinocyte cells.

FIG. 24B is a positive control showing fluorescence of Ras-induced keratinocyte cells chemically transfected with 100 Nm miRN-148b.

FIG. 24C shows fluorescence of Ras-induced Keratinocyte cells in the presence of a furan-based linker conjugated with a FAM-tagged miRN-148b and an SNP composition, where the composition has been light activated.

FIG. 25A shows a negative control of stromal media and Alizarin Red Staining D21 of hASCs cells.

FIG. 25B shows a positive control of osteogenic media and Alizarin Red Staining D21 of hASCs cells.

FIG. 25C shows a negative control of Alizarin Red Staining D21 of hASCs cells transfected with AuFe₃O₄ nanoparticles without RF activation.

FIG. 25D shows an Alizarin Red Staining D21 of hASCs cells transfected with AuFe₃O₄ nanoparticles with miRN-148b attached with a furan-based Diels-Alder linker after RF activation.

FIG. 26A is a graphical representation of SEM/EDS (scanning electron microscope/energy dispersive x-ray spectroscopy) showing mineralization levels of hASC cells with RF Activated AuFe₃O₄ nanoparticles without miRN-148b.

FIG. 26B is a graphical representation of SEM/EDS showing mineralization levels of hASC cells after RF activation of AuFe₃O₄ nanoparticles with miRN-148b attached with a furan-based Diels-Alder linker.

FIG. 27 illustrates a multistep process for the synthesis of Gold-Silver-Gold Core-Shell-Shell nanoparticles.

FIG. 28A illustrates transmission electron microscopy (TEM) images of gold seed (or core), gold-silver core-shell, and gold-silver-gold core-shell-shell nanoparticles. Scale bar=50 nm.

FIG. 28B illustrates STEM EDX mapping of the nanoparticles of FIG. 28A, for gold (Au) and silver (Ag). Scale bar=20 nm.

FIG. 29 illustrates UV-VIS absorption spectra of gold core, gold-silver core-shell, and gold-silver-gold core-shell-shell nanoparticles.

FIG. 30 illustrates an example Diels-Alder cycloaddition reaction between 6-maleimidohexanoic acid and a diene.

FIG. 31 illustrates an example synthesis of a diene with a sulfhydryl group by conjugation of cysteamine.

FIG. 32 illustrates EDC/NHS coupling chemistry for the addition of a 3′amine siRNA to the Diels-Alder linker.

FIG. 33 illustrates an example gold-silver-gold core-shell-shell light inducible delivery vehicle.

FIG. 34 illustrates light irradiation in the near-IR range (850 nm) causing photothermal cleaving of the Diels-Alder linker and subsequent siRNA payload release.

FIG. 35 illustrates an example UV-VIS absorption spectra of gold-silver-gold core-shell-shell nanoparticles (similar to FIG. 29) with the 850 nm LED used for light activation.

FIG. 36 illustrates a step function like payload release profile from gold-silver-gold core-shell-shell nanoparticles functionalized with a furan-based Diels-Alder linker with fluorescein as a payload. Nanoparticles were irradiated with an LED (850 nm) at various light energy outputs.

FIG. 37A illustrates an example embodiment of the present disclosure.

FIG. 37B illustrates a graph displaying payload release as a function of applied energy.

FIG. 37C illustrates an image of example nanoparticle in accordance with the present disclosure.

FIG. 38A illustrates a graph displaying cell number according to different treatment modalities in different cell lines.

FIG. 38B illustrates a graph displaying relative optical density for different treatment modalities.

FIG. 39A illustrates a graph displaying content of Ag localized in the kidney, lung, liver, spleen, tumor, and heart.

FIG. 39B illustrates a graph displaying relative tumor volume over time according to different treatment modalities.

FIG. 40 depicts an example implementation of the present disclosure using light activation to initiate a Retro-Diels Alder reaction.

FIG. 41A depicts an example absorbance spectrum over different wavelengths for various nanoparticle structures.

FIG. 41B depicts example TEM images for non-limiting example nanoparticle structures.

FIG. 41C depicts example STEM/EDS images for non-limiting example nanoparticle structures.

FIG. 42 depicts a graph displaying normalized intensity versus size for various compositions.

FIG. 43A depicts an example reaction scheme for synthesizing a Diels-Alder linkage.

FIG. 43B illustrates an example furan Diels-Alder (FDA) reaction.

FIG. 44A illustrates SHG intensity for Au core nanoparticles as a function of time.

FIG. 44B illustrates SHG intensity for Ag core nanoparticles as a function of time.

FIG. 44C illustrates SHG intensity for Au—Ag—Au core-shell-shell nanoparticles as a function of time.

FIG. 45 illustrates an example schematic indicating enhancement of photothermal efficiency and penetration depth for different nanoparticles (Au vs. Ag vs. Au—Ag—Au core-shell-shell nanoparticles).

FIG. 46 illustrates a bar graph depicting absolute fluorescence from released siRNA versus near infrared (NIR) light energy, showing energy required for release of therapeutic species according to some embodiments described herein.

FIG. 47A illustrates an example of cellular uptake/transfection efficiency without activation energy (“−LiGeR”).

FIG. 47B illustrates an example of therapeutic payload release with activation energy (“+LiGeR”).

FIG. 48A depicts images of intracellular release.

FIG. 48B depicts the images of intracellular release in FIG. 47A at enhanced zoom.

FIG. 49 depicts a plot of cell #upon exposure to different treatment modalities.

FIGS. 50A, 50B, 50C, and 50D illustrate bar graphs depicting two exponent negative delta delta count as an indicator of gene expression for ROCK1, TGF2, MYC, and STAT3 under the conditions mir-NC or mir-34a.

FIG. 50E depicts an image of an electrophoretic gel stained after applying samples taken under the conditions shown at the top of the gel to assess the gene on the left.

FIG. 51 illustrates a line graph showing tumor volume over days for mice provided the treatment/conditions indicated.

FIG. 52 illustrates live/dead staining of TE10 cells transfected with Au—Ag—Au core-shell-shell (CSS) particles with miRNA mimic payloads and light activated with 850 nm light to release miRNA mimics, according to some embodiments described herein. Cells were treated with either particles carrying only one miRNA mimic or with combinations of particles carrying different miRNA mimics.

FIG. 53 illustrates live/dead staining of Cal27 cells transfected with Au—Ag—Au core-shell-shell (CSS) particles with miRNA mimic payloads and light activated with 850 nm light to release miRNA mimics, according to some embodiments described herein. Cells were treated with either particles carrying only one miRNA mimic or with combinations of particles carrying different miRNA mimics.

FIG. 54 illustrates live/dead staining of SCC cells transfected with Au—Ag—Au core-shell-shell (CSS) particles with miRNA mimic payloads and light activated with 850 nm light to release miRNA mimics, according to some embodiments described herein. Cells were treated with either particles carrying only one miRNA mimic or with combinations of particles carrying different miRNA mimics.

### DETAILED DESCRIPTION

Embodiments described herein can be understood more readily by reference to the following detailed description, examples, and figures. Elements, apparatus, and methods described herein, however, are not limited to the specific embodiments presented in the detailed description, examples, and figures. It should be recognized that these embodiments are merely illustrative of the principles of this invention. Numerous modifications and adaptations will be readily apparent to those of ordinary skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10,” “from 5 to 10,” or “5-10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

It is also to be understood that the article “a” or “an” refers to “at least one,” unless the context of a particular use requires otherwise.

I. Therapeutic and/or Diagnostic Compositions

In one aspect, compositions are described herein. In some embodiments, a composition described herein comprises (a) a nanoparticle, (b) a therapeutic species, and (c) a linker joining the therapeutic species and the nanoparticle. The linker comprises a Diels-Alder cyclo-addition reaction product. As described further herein, such a composition, in some cases, is capable of delivering a therapeutic species (or other species) to a biological compartment (or other environment). Not intending to be bound by any particular theory, it is believed that such delivery can occur when externally applied light or magnetic energy “heats” the nanoparticle, followed by transfer of thermal energy from the nanoparticle to the linker. When the transferred energy is sufficient to activate (or initiate) a retro (or reverse) Diels-Alder reaction, the result is severing of the linker and decoupling and release of the therapeutic species from the nanoparticle.

The nanoparticle, the therapeutic species, and the linker of compositions of this disclosure will now be described in further detail.

### A. Nanoparticle

The nanoparticle of a composition described herein is not particularly limited and can be any nanoparticle that is not inconsistent with the objectives of this invention. For example, the nanoparticle can be a metal nanoparticle, in some embodiments, or a magnetic nanoparticle in other embodiments.

The metal nanoparticles can be formed from any suitable metal, such as any metal that can be heated by an external stimulus or signal (such as light). For example, in some instances, a nanoparticle described herein is formed from silver, gold, platinum, or a mixture or alloy thereof. In some embodiments, the metal nanoparticles are “plasmonic” metal nanoparticles, particularly metal nanoparticles having a plasmon resonant frequency at a wavelength of light suitable for use in a biological environment, such as visible light, infrared (IR) light, or near infrared (NIR). For example, the visible light in some instances is light corresponding to wavelengths from 300 nm to 700 nm; 390 nm to 700 nm; 390 nm to 600 nm; 390 nm to 500 nm; 390 nm to 450 nm; 450 nm to 700 nm (e.g., 415 nm); 500 nm to 650 nm; 550 nm to 600 nm; 500 nm to 700 nm; 550 nm to 650 nm; or 600 nm to 700 nm. The infrared light in some instances is light corresponding to wavelengths from 700 nm to 1 mm; 800 nm to 900 μm; 900 nm to 800 μm; 1000 μm to 700 μm; 900 μm to 800 μm; 700 nm to 900 μm; 700 nm to 800 μm; 700 nm to 700 μm; 700 nm to 600 μm; 700 nm to 500 μm; 700 nm to 400 μm; 700 nm to 300 μm; 700 nm to 200 μm; 700 nm to 100 μm; 700 nm to 1000 nm; 700 nm to 900 nm; or 700 nm to 800 nm. The near infrared light in some instances is light corresponding to wavelengths from 700 nm to 1 μm (1000 nm); 750 nm to 950 nm; 800 nm to 900 nm; 750 nm to 1 mm; 800 nm to 1 mm; 850 nm to 1 mm; 900 nm to 1 mm; 950 nm to 1 mm; 700 nm to 950 nm; 700 nm to 900 nm; 700 nm to 850 nm; 700 nm to 800 nm; or 700 nm to 750 nm.

In some embodiments, a nanoparticle described herein comprises a core-shell-shell nanoparticle. Such a core-shell-shell (CSS) nanoparticle can comprise a core, a first shell overlying the core, and a second shell overlying the first shell. As understood by a person of ordinary skill in the art, the first shell can fully or substantially fully (e.g., at least 95%, at least 98% or at least 99%) cover or “overcoat” the surface of the core, such that the core is contained within or “wrapped” by the first shell. Similarly, the second shell can fully or substantially fully cover or “overcoat” the surface of the first shell, such that the core and first shell are both contained within or “wrapped” by the second shell. The core, first shell, and second shell can be formed from any material not inconsistent with the objectives of the present disclosure. For example, in some cases, the core, first shell, and/or second shell comprise or are formed from a metal or a combination, alloy, or mixture of metals, where the “metals” are elemental or zero oxidation state metals, as opposed to metal ions such as may be found in a metal oxide. The metal or combination, alloy, or mixture of metals can also be the same or different for the core and one or more shells. For example, in some cases, the nanoparticle comprises a gold-silver-gold core-shell nanoparticle.

It is also possible to use a “core-shell” nanoparticle that comprises a core and a number of shells different than two. In this context, the term “core-shell” nanoparticle can be used to refer to a nanoparticle that has a core and one or more shells overcoating or overlying the core in a radial or concentric manner. For example, in some embodiments, a nanoparticle described herein can comprise a core and n sequential shells, where the nth (or outermost) shell fully or substantially fully covers or overcoats or wraps the (n−1)th shell, and the (n−1)th shell fully or substantially fully covers or overcoats or wraps the (n−2)th shell, and so on until the core is reached. In such instances, n can be any integer not inconsistent with the objectives of the present disclosure, including any integer ranging from 3 to 20 or 3 to 10. It is further to be noted that, in some embodiments, the sequential shells are in direct contact with one another (and, ultimately, with the core).

Core-shell nanoparticles described herein can be made in any manner not inconsistent with the objectives of the present disclosure, including a manner described further herein. In some cases, a core-shell nanoparticle is formed by first synthesizing the core and then synthesizing each shell sequentially, with or without purification steps in between growth of shell layers. Such synthesis can involve redox reactions of metal shell precursors or other reactions (electrochemical or otherwise) for depositing or forming a shell on a particle surface.

The core of a core-shell nanoparticle described herein can have any size described further herein. Similarly, any particular shell of a core-shell nanoparticle can have any thickness not inconsistent with the objectives of the present disclosure (where thickness is of course understood to refer to the radial dimension of the shell). For instance, in some embodiments, the outermost (e.g., second) or any other shell of the nanoparticle has an average thickness of at least 25 nm, at least 30 nm, at least 40 nm, or at least 50 nm. In some cases, the average thickness of a shell (e.g., an outermost shell) is 25-100 nm, 25-50 nm, 30-100 nm, 30-70 nm, or 50-100 nm.

Additionally, in some cases, the outermost shell or exterior surface of a nanoparticle described herein is smooth. For example, in some embodiments, the nanoparticle has a surface roughness of no greater than 20 nm, no greater than 10 nm, or no greater than 5 nm, where such surface roughness can be measured in any manner not inconsistent with the objectives of the present disclosure, such as using one or more of the following parameters: Ra, Rq, Rvi, Rpi, Rzi, or a corresponding area parameter such as Sa or Sq. Moreover, in some embodiments, the exterior surface of a nanoparticle described herein is free or substantially free of spikes or protrusions having a height of 2 nm or greater, 5 nm or greater, or 10 nm or greater.

Nanoparticles described herein can have any optical properties not inconsistent with the objectives of the present disclosure. For example, in some preferred embodiments, the nanoparticle has an optical absorbance peak in the near infrared (NIR) region of the electromagnetic spectrum, including within a specific wavelength range further described herein. For example, in some implementations, the nanoparticle has an optical absorbance peak between 800 nm and 900 nm.

Moreover, in some embodiments, the structure of the nanoparticle can be selected to provide optical properties that relate in a particular way to other optical properties of other components of a composition, system, or method described herein. The structure or characteristics of other components may also be selected in a similar manner. For example, in some embodiments, the Diels-Alder cyclo-addition reaction product of a composition described herein does not have an optical absorbance peak within 200 nm, within 150 nm, within 100 nm, within 50 nm, or within 30 nm of the optical absorbance peak of the nanoparticle. Alternatively, in other instances, the Diels-Alder cyclo-addition reaction product of a composition described herein can have an optical absorbance peak that is within 5 nm, within 10 nm, within 20 nm, within 50 nm, or within 100 nm of the optical absorbance peak of the nanoparticle. Such an optical absorbance peak of the Diels-Alder cyclo-addition reaction product, in some cases, can provide efficient energy transfer between the Diels-Alder cyclo-addition product and a plasmon of the nanoparticle, leading to efficient release of a payload as described herein.

Magnetic nanoparticles of a composition described herein can be formed from any magnetic material not inconsistent with the objectives of this disclosure. For example, in some cases, a magnetic nanoparticle is formed from a metal oxide, such as Fe₂O₃, Fe₃O₄, MnFe₂O₄, CoFe₂O₄, or NiFe₂O₄.

Further, nanoparticles described herein can have any size not inconsistent with the objectives of this disclosure. In some cases, a nanoparticle of a composition described herein has a size or diameter of 1-500 nm, 1-300 nm, 1-200 nm, 1-100 nm, 1-50 nm, 1-30 nm, 1-10 nm, 10-500 nm, 10-300 nm, 10-200 nm, 10-100 nm, 10-50 nm, 20-300 nm, 20-100 nm, 50-500 nm, 50-200 nm, or 50-100 nm in two dimensions or three dimensions. A population of nanoparticles of a composition described herein can also have an average size or diameter listed above.

Similarly, a nanoparticle of a composition described herein can also have any shape not inconsistent with the objectives of this disclosure. For example, in some cases, a nanoparticle described herein has a spherical or rod shape. Further, a nanoparticle can have a regular shape or an irregular shape.

Again not intending to be bound by theory, it is believed that the size, shape, and chemical composition of a nanoparticle described herein affect the nanoparticle's ability to be heated in response to an external stimulus described herein, such as the application of light, a magnetic field, or other source of energy.

This disclosure describes the use of photo-thermal and/or magneto-thermal properties of nanoparticles described herein, such as metal and/or magnetic nanoparticles. As understood by one of ordinary skill in the art, these properties refer to a nanoparticle's ability to convert light (photo) energy or magnetic (magneto) energy to thermal energy, which heats the nanoparticle. Thermal energy from the nanoparticle can then be transferred to the linker, resulting in severing of the linker and decoupling of the therapeutic species from the nanoparticle.

One example of the conversion of light energy to thermal energy occurs in plasmonic metallic nanoparticles. Plasmonic metallic nanoparticles resonate (e.g., form resonant plasmons) at discrete photonic wavelengths of applied light. These resonant plasmons can decay into photons, which heat the particle in direct proportion to the photo capture cross section and the quantum efficiency of the plasmon-to-phonon conversion. Thus, heating of the plasmonic nanoparticles occurs primarily when a wavelength of the applied light matches the unique resonant frequency of the nanoparticles and forms a resonant plasmon.

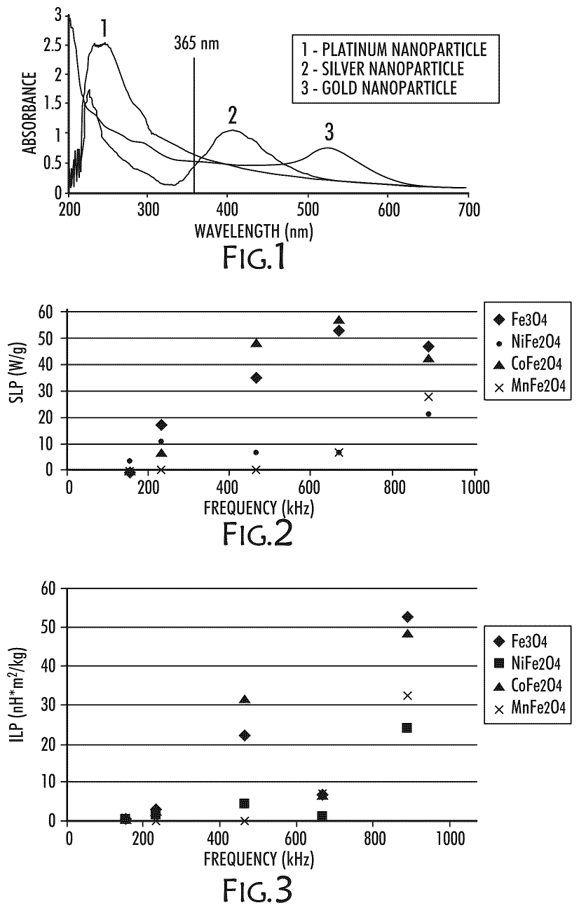

A main determinate of resonant frequency in these plasmonic metal nanoparticles is composition. For example, silver nanoparticles have a resonant frequency with a local maximum at about 420 nm, whereas gold nanoparticles resonate at about 535 nm, and platinum nanoparticles resonate at about 215 nm, as illustrated in FIG. 1.

Another determinant of resonance frequency is morphology. As an example, nanorods have multiple resonant frequencies correlating to transverse and longitudinal modes. The longitudinal modes provide red shifted resonance frequencies in the near infrared (NIR) region of the spectrum. The transverse modes have much shorter wavelengths, typically found in the optical or visible region of the electromagnetic spectrum.

Due to the discrete resonant frequencies of the plasmonic nanoparticles described herein, heating of the nanoparticles, and, as a result, severing of the linker and decoupling of the therapeutic species from the nanoparticle, can be controlled. In general, for heating of plasmonic nanoparticles to occur to a degree sufficient to sever a linker in a manner described herein, the frequency of applied light must match or substantially match a resonance frequency of the plasmonic nanoparticle.

In some embodiments, compositions described herein comprise a plurality of differing plasmonic nanoparticles. More particularly, in some cases, the differing plasmonic nanoparticles have different compositions and/or morphologies. Such nanoparticles can thus have different resonance frequencies. In these embodiments, decoupling of the therapeutic species from the nanoparticle (e.g., in a biological compartment) can be spatiotemporally controlled by using different color (frequency and wavelength) light for stimulation of the plurality of nanoparticles at different times or in different locations (e.g., different locations within the biological compartment). The different light frequencies can each match or substantially match at least one of the resonance frequencies of the different plasmonic nanoparticles.

Another example of the above-mentioned conversion of incident energy, external energy, or activating energy to thermal energy can occur when magnetic nanoparticles are used. Magnetic nanoparticles can convert magnetic energy from an applied alternating magnetic field to thermal energy that heats the nanoparticles. Magnetic nanoparticles, like plasmonic metal nanoparticles, can be tuned for specific loss power or to have a discrete frequency response based on their chemical composition and morphology. As understood by one of ordinary skill in the art, the specific loss power (SLP) is a figure of merit for conversion of magnetic energy from an applied alternating field into thermal energy. The higher the SLP, the greater the efficiency of conversion at a particular frequency. Depending on the frequency regime, particles can exhibit distinct SLP values based on the particles' compositions and morphology. For example, as shown in FIG. 2A, CoFe₂O₄ magnetic nanoparticles described herein, in some embodiments, had a higher SLP value compared to MnFe₂O₄, NiFe₂O₄, and Fe₃O₄ magnetic nanoparticles at a frequency of 450 and 650 kHz. Again, with reference to FIG. 2A, Fe₃O₄ nanoparticles had a higher SLP value, in some embodiments, at a frequency of 200 kHz than the other nanoparticles. In some embodiments, the magnetic nanoparticles described herein have an SLP value greater than 20, 25, 30, or 40 W/g at an applied alternating magnetic field frequency of 850 kHz. For example, at an applied alternating magnetic field frequency of 850 kHz, the magnetic nanoparticles have an SLP value of 20 W/g to 80 W/g; 20 W/g to 70 W/g; 20 W/g to 60 W/g; 20 W/g to 50 W/g; 20 W/g to 40 W/g; 20 W/g to 30 W/g; 30 W/g to 80 W/g; 40 W/g to 80 W/g; 50 W/g to 80 W/g; 60 W/g to 80 W/g; 70 W/g to 80 W/g; 25 W/g to 70 W/g; 30 W/g to 60 W/g; or 35 W/g to 50 W/g. Moreover, in some cases, magnetic nanoparticles described herein have an SLP value greater than 2, 5, or 10 W/g at an applied alternating magnetic field frequency of 200 kHz. For example, at an applied alternating magnetic field frequency of 200 kHz, the magnetic nanoparticles have an SLP value of 1 W/g to 20 W/g; 1 W/g to 15 W/g; 1 W/g to 10 W/g; 1 W/g to 9 W/g; 1 W/g to 8 W/g; 1 W/g to 7 W/g; 1 W/g to 6 W/g; 1 W/g to 5 W/g; 1 W/g to 4 W/g; 1 W/g to 3 W/g; 2 W/g to 12 W/g; 2 W/g to 12 W/g; 3 W/g to 12 W/g; 4 W/g to 12 W/g; 5 W/g to 12 W/g; 6 W/g to 12 W/g; 7 W/g to 12 W/g; 8 W/g to 12 W/g; 9 W/g to 12 W/g; or 10 W/g to 12 W/g.

Intrinsic loss power (ILP), as understood by one of ordinary skill in the art, is another indicator of heating efficiency at a given magnetic frequency. Higher ILP values correspond to greater heating efficiency. ILP data for nanoparticles according to some embodiments described herein are provided in FIG. 2B. In some instances, ILP values for the nanoparticles described herein range from 0.1 to 12 nHm²/kg, 0.1 to 11 nHm²/kg, 0.1 to 10 nHm²/kg, 0.1 to 9 nHm²/kg, 0.1 to 8 nHm²/kg, 0.1 to 7 nHm²/kg, 0.1 to 6 nHm²/kg, 0.1 to 5 nHm²/kg, 0.1 to 4 nHm²/kg, 0.23-3.1 nHm²/kg, 0.1 to 2 nHm²/kg, or 0.1 to 1 nHm²/kg.

### B. Therapeutic Species

The therapeutic species of a composition described herein is not particularly limited. Any therapeutic species not inconsistent with the objectives of this disclosure can be used. Moreover, the therapeutic species can be any species useful for treating a disease or condition of a patient, including treating a human patient in vivo. For example, in some cases, a therapeutic species of a composition described herein is a small molecule, a nucleic acid, a peptide, a protein, or any combination thereof. The nucleic acid can comprise a plasmid, a small interfering RNA (“siRNA”), a micro-RNA (“miRNA”), an miRNA mimic, or any combination thereof. As understood by those of ordinary skill in the art, miRNA mimics are chemically modified double-stranded RNAs that mimic endogenous miRNAs and enable miRNA functional analysis by up-regulation of miRNA activity. In some embodiments, a therapeutic species described herein is an RNA sequence. The term small molecule is understood by those of ordinary skill in the art to comprise a hydrocarbon-based compound having a molecular weight between 100 daltons to 1000 daltons; 100 daltons to 900 daltons; 100 daltons to 800 daltons; 100 daltons to 700 daltons; 100 daltons to 600 daltons; 100 daltons to 500 daltons; 100 daltons to 400 daltons; 100 daltons to 300 daltons; 100 daltons to 200 daltons; 200 daltons to 900 daltons; 250 daltons to 800 daltons; 300 daltons to 700 daltons; 350 daltons to 600 daltons; 400 daltons to 500 daltons; 200 daltons to 1000 daltons; 300 daltons to 1000 daltons; 400 daltons to 1000 daltons; 500 daltons to 1000 daltons; 600 daltons to 1000 daltons; 700 daltons to 1000 daltons; 800 daltons to 1000 daltons; or 900 daltons to 1000 daltons. The small molecule can also comprise a hydrocarbon-based compound having additional heteroatoms, such as O, N, S, B, P, or any combination thereof. Additionally, the small molecule can be saturated or unsaturated, having single, double, or triple bonds. The small molecule can also in some instances be linear or cyclic (both aromatic or nonaromatic).

Additionally, in some instances, a therapeutic species of a composition described herein can be a theranostic species. Such a species can be used to diagnose a disease or condition, as well as treat the disease or condition. Non-limiting examples of theranostic species include therapeutic species described above, wherein the therapeutic species is also luminescent (e.g., fluorescent or phosphorescent), radioactive, MRI active, or otherwise capable of being imaged or tracked, including in a human patient in vivo.

In still other cases, a therapeutic or theranostic species described herein can be replaced or at least partially replaced with a diagnostic species or an imaging agent. Such a diagnostic species can be used to diagnose a disease or condition rather than treat the disease or condition. For example, in some embodiments, the diagnostic agent can be an antibody specific to biomarkers expressed in a cell. For instance, the diagnostic agent could be an antigen specific to a particular cell type or disease, such as a prostate specific antigen (PSA) to diagnose the presence of prostate cancer.

A contrast agent, in some cases, comprises a computed tomography (CT) contrast agent such as a radiocontrast agent or iodinated contrast agent. In some instances, a contrast agent comprises a magnetic resonance imaging (MRI) contrast agent, such as a positive magnetic resonance (T1) contrast agent. In some embodiments, such a positive contrast agent includes a chemical species comprising gadolinium or another lanthanide, such as gadolinium chloride. Moreover, a contrast agent suitable for use in some embodiments described herein can be a molecular contrast agent or a particulate contrast agent. A contrast agent may also be a nanoparticulate material. In some cases, a contrast agent comprises superparamagnetic iron oxide (SPIO) such as Feraheme or Ferumoxytol, gold manganese, or gadolinium. Other contrast agents may also be used.

An imaging agent, in some embodiments, comprises a luminescent species, such as a fluorescent species or phosphorescent species. In some instances, an imaging agent comprises an organic fluorophore or dye such as a rhodamine, coumarin, or cyanine (such as NIR-797). In some embodiments, an imaging agent comprises a luminescent biomolecule such as green fluorescent protein (GFP) or plasmid DNA vector encoding yellow fluorescent protein (pEYFP-N1). An imaging agent may also comprise an organic fluorophore or dye conjugated to a biomolecule, such as rhodamine conjugated bovine serum albumin (BSA-rhodamine). In still other cases, an imaging agent comprises an inorganic material such as a semiconductor nanocrystal or quantum dot, which may include a Group II-VI semiconductor nanocrystal (such as CdSe) or a Group III-V semiconductor nanocrystal (such as InP or InAs).

Moreover, an imaging agent described herein can emit light having any wavelength or luminescence profile not inconsistent with the objectives of the present disclosure. For instance, in some embodiments, an imaging agent emits light having a wavelength centered in the near-infrared region of the electromagnetic spectrum. An imaging agent may also emit light having a wavelength centered in the visible region or the non-near-infrared region of the electromagnetic spectrum. In some cases, for example, an imaging agent described herein has an emission profile centered at a wavelength between 400 nm and 700 nm, between 500 nm and 650 nm, between 600 nm and 900 nm, between 700 nm and 900 nm, between 750 nm and 850 nm, between 800 nm and 1100 nm, between 1100 nm and 1400 nm.

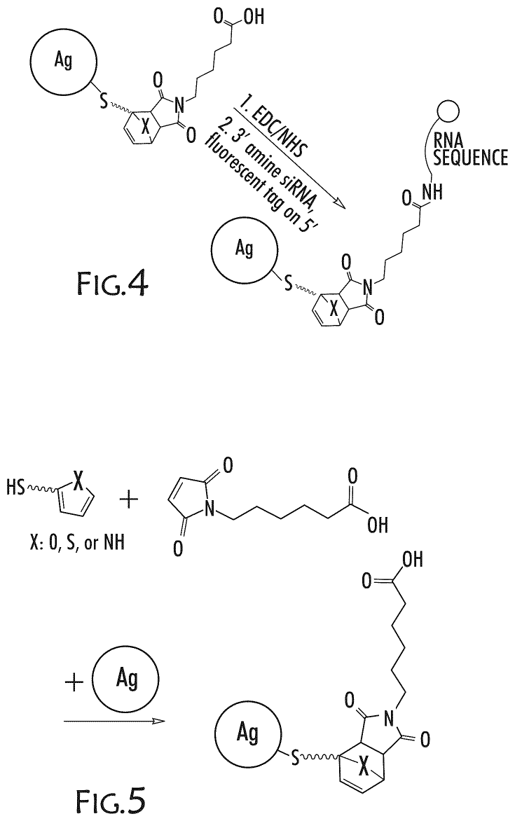

Further, a therapeutic species (or theranostic or diagnostic species or imaging agent) described herein can be connected, attached, or bonded to a linker in any manner not inconsistent with the objectives of this disclosure. In some embodiments, for instance, the therapeutic species is connected to the linker via a covalent bond. For example, an amine-terminated therapeutic species (e.g., an amine-terminated nucleic acid) can be covalently bonded to a linker by reacting the amine-terminated nucleic acid with a carboxy-terminated end of a linker via EDC (1-Ethyl-3-(3-dimethylaminopropyl)-carbodiimide) coupling or other carbodiimide coupling. Carbodiimide coupling is described in Dunetz J, Magano J, Weisenburger G., “Large-Scale Applications of Amide Coupling Reagents for the Synthesis of Pharmaceuticals,” Org. Process Res. Dev. 2016, 20, 140-177, which is incorporated by reference in its entirety herein. FIG. 4 and FIG. 5 also illustrate such linkages. Specifically, FIG. 4 shows an exemplary EDC-coupling reaction of an amine-terminated RNA sequence being covalently bound to a carboxy-terminated end of a linker.

In some embodiments, the composition described herein is for use in tissue regeneration. In this instance, the therapeutic species is a tissue regenerative species (i.e., “tissue regenerator”). The tissue regenerative species can comprise an osteogenic modulator, a chondrogenic modulator, an endotheliologenic modulator, or a myogenic modulator. Exemplary embodiments of osteogenic modulators can comprise, simvastatin, strontium ranelate, miRNA 26a, 148b, 27a, and 489, or any osteogenic modulator not inconsistent with the objectives of this disclosure. Exemplary embodiments of chondrogenic modulators comprise, miR-9, miR-140 and miR-30A, although the chondrogenic modulator described herein can be any chondrogenic modulator not inconsistent with the objectives of this disclosure. Exemplary embodiments of endotheliogenic modulators comprise miR-210, miR-195, miR-155, miR-106b, miR-93, and miR-25, although the endotheliogenic modulator described herein can be any endotheliogenic modulator not inconsistent with the objectives of this disclosure. Exemplary embodiments of myogenic modulators comprise miR-206, miR-1, siGDF-8, miR-133, miR-24 and miR-16 although the myogenic modulator described herein can be any myogenic modulator not inconsistent with the objectives of this disclosure.

In some embodiments, the composition described herein is for use in cancer chemotherapy. In this instance, the therapeutic species can comprise an anti-cancer or anti-tumor agent (hereinafter generally called “anti-cancer”). Exemplary embodiments of anti-cancer species comprise Paclitaxil, Afatinib, Dimaleate, Bortezomib, Carfilzomib, Doxorubicin, Fluorouracil, miRNA-148b, -135, -124, -101, -29c, -15a, and -34 (MRX34) mimics or any anti-cancer agent not inconsistent with the objectives of this disclosure.

More particularly, in certain embodiments a composition described herein can be used for treatment/chemotherapy for brain tumors such as glioblastoma multiform (GBM). In some such implementations, the therapeutic species can include one or more miRNA species. Exemplary miRNA that can be included in example implementations are described in Table 1 below.

TABLE 1
miRNA Species

| Name | Sequence |
|---|---|
| Hsa-miR-34a-5p | 5′ 6-FAM 2′Ome |
| | UGGCAGUGUCUUAGCUGGUUGUC6- |
| | NHs 3′ |
| Hsa-miR-7-5p | 5′ 6-FAM 2′Ome |
| | UGGAAGACUAGUGAUUUUGUUGUU- |
| | NHs 3′ |
| Hsa-miR-218-5p | 5′ 6-FAM 2′Ome |
| | UUGUGCUUGAUCUAACCAUGU-NHs 3′ |
| Hsa-miR-148b-3p | 5′ 6-FAM 2′Ome |
| | UCAGUGCAUCACAGAACUUUGU-NHs |
| | 3′ |
| Hsa-miR-433-3p | 5′ 6-FAM 2′Ome |
| | AUCAUGAUGGGCUCCUCGGUGU-NHs |
| | 3′ |
| Hsa-miR-181a-5p | 5′ 6-FAM 2′Ome |
| | AACAUUCAACGCUGUCGGUGAGU- |
| | NHs 3′ |

In some embodiments, a composition described herein can include one or a combination of more than one miRNA species (or other therapeutic species) described herein. For instance, combinatorial therapies may be used in certain implementations. Some compositions in accordance with the present disclosure can include a first metal nanoparticle linked to a first miRNA and a second metal nanoparticle linked to a second miRNA, where the first miRNA is different from the second miRNA. It is also possible for a single nanoparticle described herein to be linked to a plurality of differing miRNA species (or other therapeutic species), such as a first miRNA species and a second miRNA species that differs from the first miRNA species. In some preferred embodiments, for example, a combination of first and second miRNA species are used, as described in Table 2 below.

TABLE 2
Combinations of miRNA Species

| First miRNA Species | Second miRNA Species |
|---|---|
| miR34a | miR218a |
| miR148b | miR218a |
| miR34a | miR148b |
| miR181a | miR148b |
| miR181a | miR34a |
| miR181a | miR218a |

### C. Linker

The linker of a composition described herein is not particularly limited and can be any linker that is not inconsistent with the objectives of this disclosure. As described above, the linker comprises a Diels-Alder cyclo-addition product. As understood by one of ordinary skill in the art, a Diels-Alder reaction is a conjugate addition reaction of a conjugated diene with a dieneophile. Moreover, the dienophile can comprise an ethyleneically unsaturated moiety. For instance, in some cases, the dienophile is a substituted or unsubstituted alkene or alkyne. In some embodiments, the Diels-Alder cyclo-addition product of a composition described herein is a reaction product of a dienophile with a furan, thiophene, or a pyrrole. An exemplary Diels-Alder reaction of a furan, thiophene, or pyrrole (each being exemplary dienes) with 6-maleimide hexanoic acid (an exemplary dienophile) is shown for example in FIG. 5.

In addition, example dienes according to the present disclosure can further include an additional functional group or reactive moiety, such as a sulfhydryl group. Non-limiting examples of dienes containing a sulfhydryl group include the following: 2-Thiophenemethanethiol, 3-Thiophenemethanethiol, 2-Methylthiophene-3-thiol, 1-(2-Thienyl)ethanethiol, 5-Methylthiophene-2-thiol, 5-Methyl-3-thiophenethiol, 5-Ethylthiophene-2-thiol, 2,5-bis(mercaptomethyl)thiophene, 2,5-dimethylthiophene-3-thiol, Thiol-decanethiol, 1-(2-Methylthiophen-3-yl)sulfanylethanethiol, 2-Thiopheneethanethiol, 5-Methylthiophene-2-methanethiol, 5-ethylthiophene-3-thiol, 1-thiophen-2-ylpropane-1,1-dithiol, 1-thiophen-2-ylpropane-2-thiol, 5-methylthiophene-2,3-dithiol, 5-mercapto-2-thienylbutane, Furfuryl mercaptan, 5-Methyl-2-furanmethanethiol, 2-(1-Mercaptoethyl)furan, (3-Methylfuran-2-yl)methanethiol, 2-Methyl-3-furanthiol, 5-Methyl-3-furanthiol, (2-Methylfuran-3-yl)methanedithiol, 2-Furanethanethiol, [5-(Sulfanylmethyl)furan-2-yl]methanethiol, furan-2-ylmethanedithiol, 1-(2-Methylfuran-3-yl)ethanethiol, 3-(furan-2-yl)propane-1-thiol, 1-(Furan-2-ylmethylsulfanyl)butane-2-thiol, 2-[2-[2-(Furan-2-yl)-1,3-dithiol-4-yl]ethylsulfanyl]ethanethiol, 1-(Furan-2-yl)ethane-1,2-dithiol, (3-Ethylfuran-2-yl)methanethiol, (5-Ethylfuran-2-yl)methanethiol, 2-[1-(disulfanyl)ethyl]furan, 1-(Furan-2-yl)propane-2-thiol, 2-(Furan-2-yl)ethane-1,1-dithiol, 1-(5-Methylfuran-2-yl)ethane-1-thiol, (2-methylfuran-3-yl)methanethiol, 2-[2-(Disulfanyl)ethyl]furan, 4-(Furan-2-yl)butane-2-thiol, 2-Furanpropanethiol, 2-(5-methylfuran-2-yl)ethanethiol, 2-(disulfanylmethyl)furan, 3-(furan-2-ylmethylsulfanyl)propane-1-thiol, 2-(furan-2-ylmethoxy)ethanethiol, 1H-pyrrol-2-ylmethanethiol, (3-Methyl-1H-pyrrol-2-yl)methanethiol, 2-(2-Methyl-1H-pyrrol-3-yl)ethanethiol, 1-(1H-Pyrrol-2-ylmethyl)piperidine-3-thiol, 5-Ethyl-1H-pyrrole-3-thiol, 2-Methyl-1H-pyrrole-3-thiol, 1-(2-Ethyl-1H-pyrrol-3-yl)ethanethiol, 5-Methyl-1H-pyrrole-3-thiol, 2-(Disulfanylmethyl)-1H-pyrrole, 2-(1H-Pyrrol-2-yl)propane-2-thiol, 1-(1H-Pyrrol-2-yl)ethanethiol, 12-(1H-Pyrrol-2-yl)dodecane-1-thiol, 8-(1H-Pyrrol-2-yl)octane-1-thiol, 11-(1H-Pyrrol-2-yl)undecane-1-thiol, 2-(1H-Pyrrol-2-yl)ethanethiol, 1-Cyclopropyl-2-(1H-pyrrol-2-yl)ethanethiol, 4-(1H-Pyrrol-2-yl)butane-1-thiol, 3-(1H-Pyrrol-2-yl)propane-1-thiol, and 2-Methyl-2-(1H-pyrrol-2-yl)propane-1-thiol.

Such dienes having a sulfhydryl group or another additional functional group (where an “additional” functional group refers to a functional group that is not needed in the Diels-Alder cyclo-addition reaction but can be used to react separately with another species, such as to form a covalent bond with a therapeutic species and/or a nanoparticle surface described herein) are preferred in some embodiments described herein. Additional functional groups, which are not particularly limited, can include hydroxyl groups or carboxyl groups, as well as sulfhydryl or thiol groups. In some embodiments of a composition described herein, therefore, a linker is covalently bonded to the nanoparticle and/or to the therapeutic species through an R—S—X bond, where R is alkyl or another organic substituent bonded to sulfur through a carbon atom and X is the nanoparticle or the therapeutic species.

As stated above, in some implementations, the diene can include a carboxyl group. Several non-limiting examples of dienes including a carboxyl group include the following: 1H-pyrrole-2-carboxylic acid, 1H-pyrrole-3-carboxylic acid, 3,5-dimethyl-1H-pyrrole-2-carboxylic acid, 1,5-dimethyl-1H-pyrrole-2-carboxylic acid, 2,4,5-trimethyl-1H-pyrrole-3-carboxylic acid, 5-phenyl-1H-pyrrole-2-carboxylic acid, 2,4-dimethyl-1H-pyrrole-3-carboxylic acid, 2,5-dimethyl-1H-pyrrole-3-carboxylic acid, 3-methyl-1H-pyrrole-2-carboxylic acid, 5-(3,4-dimethylphenyl)-2-methyl-1H-pyrrole-3-carboxylic acid, 1-methyl-1H-pyrrole-2-carboxylic acid, 2-methyl-1H-pyrrole-3-carboxylic acid, furan-2-carboxylic acid, furan-3-carboxylic acid, 2-(furan-2-yl)acetic acid, 3-(5-methylfuran-2-yl)propanoic acid, 5-ethylfuran-2-carboxylic acid, 5-isobutyl-2-methylfuran-3-carboxylic acid, 4,5-dimethylfuran-2-carboxylic acid, thiophene-2-carboxylic acid, 4,5-dimethylthiophene-2-carboxylic acid, 3-methylthiophene-2-carboxylic acid, 5-methylthiophene-2-carboxylic acid, 5-phenylthiophene-2-carboxylic acid, 2-(thiophen-2-yl)acetic acid, thiophene-3-carboxylic acid, 2-(thiophen-3-yl)acetic acid, 5-ethylthiophene-2-carboxylic acid, and 5-methyl-4-phenylthiophene-3-carboxylic acid.

Dienes having a sulfhydryl group can be prepared in any manner not inconsistent with the objectives of the present disclosure, including using a protocol described in the specific Examples below. It is also possible, in place of cysteamine, to form such a modified diene using one or more of the following reactants: 3-Aminopropanethiol, 1-Aminoethanethiol, Thiocysteamine, 2-(Disulfanyl)ethanamine, 1-Aminoethane-1,2-bisthiol, (2-Aminoethyldisulfanyl)methanethiol, 2,4-Bis-(4-methoxyphenyl)-1,3-dithia-2,4-diphosphetane 2,4-disulfide, 4-aminobutane-1-thiol, 1-Aminopropane-1-thiol, 1-Aminopropane-2-thiol, 3-Aminopropane-1,2-dithiol, 3-(trisulfanyl)propan-1-amine, 1-aminopropane-1,3-dithiol, 5-Aminopentane-1-thiol, 1-Amino-2-butanethiol, 1-Aminobutane-3-thiol, 1,2-Aminobutanethiol, 1-Aminobutane-2,3-dithiol, 1-Aminobutane-1,3-dithiol, 1-Aminobutane-1,4-dithiol, 4-Aminobutane-1,1-dithiol, 4-(Disulfanyl)butan-1-amine, 1-Aminobutane-1,2-dithiol, 4-Aminobutane-1,2-dithiol, 4-Aminobutane-1,3-dithiol, 6-Aminohexane-1-thiol, 2-Aminohexane-1-thiol, 5-aminohexane-2-thiol, 6-Aminohexane-1,3-dithiol, 6-Aminohexane-1,1-dithiol, 6-aminohexane-3-thiol, 6-aminohexane-2-thiol, 5-aminohexane-1-thiol, 4-Aminohexane-1-thiol, 7-aminoheptane-1-thiol, 7-Aminoheptane-3-thiol, 1-Aminoheptane-1-thiol, 1-Aminopentane-3-thiol, 5-Aminopentane-2-thiol, 1-Aminoheptane-4-thiol, 1-Aminohexane-3-thiol, 3-aminohexane-1-thiol, 3-Aminohexane-2-thiol, 5-Aminopentane-1-thiol, 6-Aminoheptane-1-thiol, 6-Aminohexane-1-thiol, 8-Amino-1-octanethiol, 9-Aminononane-1-thiol, 9-Aminononane-1,1-dithiol, 10-Aminodecane-1-thiol, 11-Amino-1-undecanethiol, 12-Aminododecane-1-thiol, and 15-Aminopentadecane-1-thiol.

Exemplary dienophiles suitable for use in some embodiments described herein include the following: 2-Maleimidoacetic acid, 3-Maleimidopropionic acid, 3-Maleimidobenzoic acid, 3-(2,5-Dioxopyrrol-1-yl)hexanoic acid, 4-Maleimidobutyric acid, 4-Maleimidobenzoic acid, 4-(2,5-Dioxopyrrol-1-yl)hexanoic acid, 4-(2,5-dioxo-2,5-dihydro-pyrrol-1-yl)-benzoic acid, 5-Maleimidopentanoic acid, 6-Maleimidohexanoic acid, 6-(3-methyl-2,5-dioxopyrrol-1-yl)hexanoic acid, 6-(2,5-dioxopyrrol-1-yl)-2-methylhexanoic acid, 6-(2,5-Dioxopyrrol-1-yl)-4-methylhexanoic acid, 7-(2,5-Dioxo-2,5-dihydro-1H-pyrrol-1-yl)heptanoic acid, 9-(2,5-dioxopyrrol-1-yl)nonanoic acid, 10-(2,5-dioxopyrrol-1-yl)decanoic acid, 11-Maleimidoundecanoic acid, 13-(2,5-Dioxo-2,5-dihydro-1H-pyrrol-1-yl)tridecanoic acid, N-(Carboxyheptyl)maleimide, N-(4-Carboxy-3-hydroxyphenyl)maleimide, 1,1′-(Methylenedi-4,1-phenylene)bismaleimide, and α-Maleimidyl-ω-Carboxyl Poly (ethylene glycol).

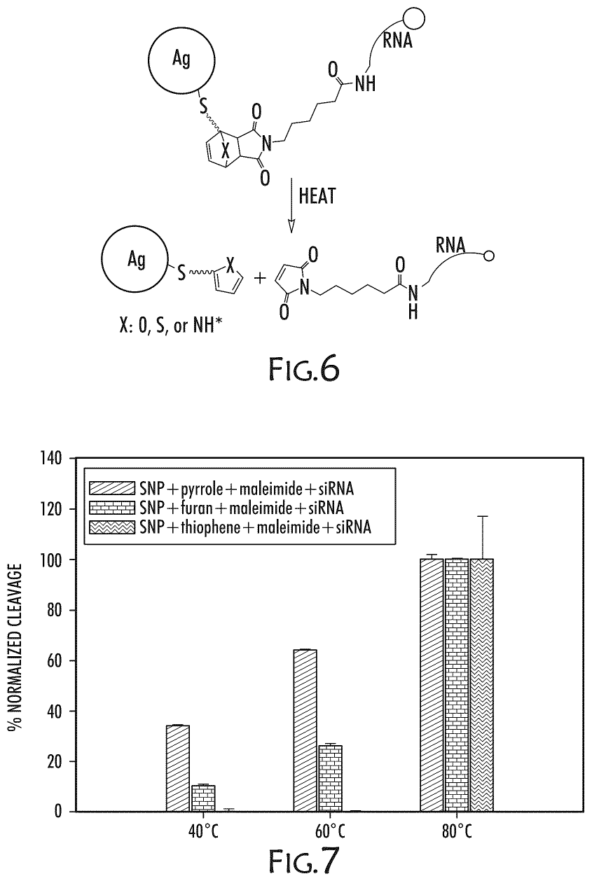

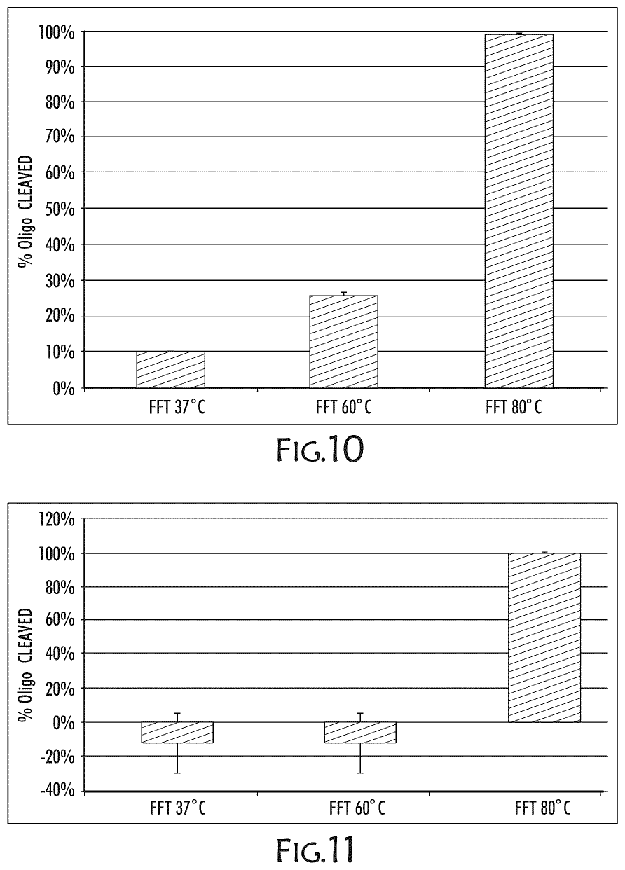

As described further herein, the Diels-Alder cyclo-addition product in the linker can undergo a retro (or reverse) Diels-Alder reaction. This retro (or reverse) Diels-Alder reaction breaks up the cyclo-addition product formed by a (forward) Diels-Alder reaction into the reaction precursors that originally formed the cyclo-addition product in the forward Diels-Alder reaction. This breaking up or cleaving of the Diels-Alder cyclo-addition product in the linker results in decoupling of the therapeutic species and the nanoparticle, as illustrated in FIG. 6. For different Diels-Alder cyclo-addition products, the retro (or reverse) Diels-Alder reaction will occur or be initiated at different temperatures (i.e., different activation temperatures). Thus, by varying the Diels-Alder cyclo-addition product in the linker group, breaking up or cleaving of the Diels-Alder cyclo-addition product (and concomitant decoupling of the therapeutic species and the nanoparticle) can be “tuned” to occur at different temperatures. For example, the activation temperature of the pyrrole-based linker group in FIG. 7 was measured and found to be 40° C., the furan-based linker group was measured and found to be 60° C., and the activation temperature of the thiophene-based linker group in FIG. 7 was found to be around 80° C. It was also found that cleavage of a furan-based linker occurs faster at lower temperatures than the thiophene-based linker, as seen for example in FIGS. 10 and 11. FIG. 10 shows temperature dependency of a retro Diels-Alder reaction (and release of a fluorescent si-RNA) for the furan-based linker group (connected to a nanoparticle) after 1 hour of heating at 37° C., 60° C., and 80° C., where 25% cleavage of the linker is observed at 60° C. FIG. 11 shows temperature dependency of a retro Diels-Alder reaction (and release of the fluorescent si-RNA) for a thiophene-based linker group (connected to a nanoparticle) after 2 hours of heating at 37° C., 60° C., and 80° C., where cleavage of the linker is not observed until 80° C.

In some embodiments, the activation temperature of the retro (or reverse) Diels-Alder reaction of a linker described herein is between 30° C. and 170° C., between 30° C. and 150° C., between 40° C. and 85° C., between 45° C. and 100° C., between 50° C. and 80° C., or between 55° C. and 70° C.

As described further herein, the activation temperature of a retro Diels-Alder reaction can be reached, at least in part, by transfer of thermal energy from the nanoparticle to the linker, raising the temperature of the linker or of the immediate environment of the linker. Additionally, in some embodiments, the Diels-Alder cyclo-addition reaction product of a composition described herein has a forward reaction activation energy and a backward reaction activation energy. The forward reaction activation energy can be the activation energy (e.g., from the Arrhenius equation) for the Diels-Alder reaction that forms the cyclo-addition reaction product. Similarly, the backward reaction activation energy can be the activation energy for the retro Diels-Alder reaction. In some embodiments described herein, the backward reaction activation energy is at least 1.5 times or at least 1.2 times the forward reaction activation energy. In some cases, the backward reaction activation energy is 0.5-2 times, 0.5-1.5 times, 0.5-1 times, 0.5-0.9 times, 0.5-0.7 times, 1-1.2 times, 1-1.5 times, 1.2-2 times, 1.5-3 times, or 1.5-5 times the forward reaction activation energy. Moreover, in some implementations, the backward reaction activation energy is greater than the thermal energy provided by aqueous surroundings of the nanoparticle at a temperature of 310K. In still other instances, the backward reaction activation energy (in kJ/mol) is less than a maximum thermal energy (in kJ) of the nanoparticle generated by absorption by the nanoparticle of a mole of photons at the NIR optical absorbance peak of the nanoparticle. Compositions having such properties as described above, in some cases, can be both stable in environments such as in vivo environments and also able to be activated or cleaved (such that payload release occurs) at relatively low energies or at energies (e.g., provided by light irradiation or magnetic field exposure) that provide one or more clinical advances, such as precise payload release control.

Additionally, in some embodiments, compositions described herein can include two or more different linkers, wherein the linkers comprise different Diels-Alder cycloaddition products. The use of a plurality of differing linkers can allow for decoupling of therapeutic species and nanoparticles at different temperatures. In this manner, the amount and/or type of therapeutic species delivered to a biological compartment can be controlled. For example, if different therapeutic species are attached to different nanoparticles via different linkers, then the release of the different therapeutic species can be controlled by heating the population of different nanoparticles to the retro (or reverse) Diels-Alder activation temperature of one linker group, but not the other. Such heating can result in release of one therapeutic species, but not the other.

A linker described herein can be connected, attached, or bonded to a therapeutic species and/or nanoparticle in any manner not inconsistent with the objectives of this disclosure. For example, in some cases, a linker is bonded to a nanoparticle via one or more metal-ligand bonds (such as shown in FIGS. 4-6 (for a metal nanoparticle) and for magnetic nanoparticles (not shown)). Such bonds can be covalent or non-covalent. For instance, in some embodiments, a linker is attached to a nanoparticle via one or more ionic bonds.

II. Methods of Delivering a Therapeutic and/or Diagnostic Species to an Environment

In another aspect, methods of delivering a therapeutic and/or diagnostic species to an environment are described herein. In some instances, such a method comprises disposing a composition described herein in an environment, such as a biological compartment. Any composition described hereinabove in Section I can be used. For example, in some cases, the composition comprises a metal or magnetic nanoparticle and a linker joining the nanoparticle to the therapeutic species, wherein the linker comprises a Diels-Alder cyclo-addition product. A method described herein can further comprise initiating a retro Diels-Alder reaction to decompose the Diels-Alder cyclo-addition product of the composition. In this manner, the linker can be severed and the therapeutic species can be decoupled from the nanoparticle, including in a desired location. Specific steps of methods described herein will now be described in further detail.

### A. Disposing a Composition in an Environment

A composition described herein can be disposed in an environment in any manner not inconsistent with the objectives of this disclosure. Moreover, the environment can be any environment not inconsistent with the objectives of this disclosure. In some cases, for example, the environment is a biological environment or compartment. Such an environment or compartment, in some instances, comprises or consists essentially of a cell, tissue, organ, or body cavity of a living mammal, such as a human. Further, in some embodiments, a composition described herein is disposed in a biological compartment by injecting the composition into the compartment directly or indirectly, such as can be achieved by subcutaneous injection or injection into vasculature of a mammal.

A composition described herein can also be disposed in a biological compartment of a mammal by topical application of the composition to a surface of skin or other surface of the mammal. In some cases, a composition described herein is disposed in a biological compartment by diffusion of the composition into the compartment, or by oral ingestion. Other methods of disposing a composition in an environment can also be used.

### B. Initiating a Retro Diels-Alder Reaction

The step of initiating a retro Diels-Alder reaction to decompose the Diels-Alder cycloaddition product, thereby severing the linker and decoupling the therapeutic species from the magnetic or thermal-activated nanoparticle, can be performed or carried out in any manner not inconsistent with the objectives of this disclosure. Severing the linker and decoupling the therapeutic species releases the therapeutic species. In some embodiments when the linker is severed in a biological compartment, the therapeutic species is released into the biological compartment. In some embodiments, the step of initiating a retro Diels-Alder reaction comprises heating the nanoparticle of a composition described herein to an activation temperature of the retro Diels-Alder reaction.

As described above, heating the nanoparticle can be achieved in a different manner based on the type of nanoparticle (e.g., metal nanoparticle or magnetic nanoparticle). For a metal nanoparticle, for instance, heating can be achieved by irradiating the nanoparticle with light comprising a wavelength (or frequency) that matches or corresponds to a resonant frequency of the nanoparticle. Irradiating a nanoparticle with such light can result in the formation of resonant plasmons. These resonant plasmons can decay into phonons, which heat the particle, as described above.

In such embodiments wherein a metal nanoparticle is used, the applied light can have an average wavelength in the visible, infrared, or near infrared spectrums, as previously described above. Such wavelengths can be especially advantageous, in some cases, when used in sub-toxic intensities.

In some cases wherein exposure to light is used to heat a nanoparticle and/or release a payload, NIR in particular is used. In some such embodiments, NIR light has a wavelength distribution centered around a central exposure wavelength that is within 30 nm, within 20 nm, or within 10 nm of the NIR optical absorbance peak of the nanoparticle. Moreover, in some instances, the nanoparticle absorbs at least a portion of the energy of the NIR light in a surface plasmon resonance process to provide an absorbed plasmon resonance energy. Further, in some embodiments, the absorbed plasmon resonance energy is at least partially converted to thermal energy of the nanoparticle, thereby increasing a temperature of the nanoparticle. Additionally, increasing the temperature of the nanoparticle can result in heating the nanoparticle to the activation temperature of the Diels-Alder reaction.

In some embodiments described herein, the Diels-Alder cyclo-addition product does not absorb more than 3%, more than 5%, more than 10%, or more than 15% of incident photons having a wavelength within the wavelength distribution of the NIR light. In this manner, “competition” for photons between the Diels-Alder cyclo-addition product and the nanoparticle can be avoided or minimized. It is to be understood that the reference to NIR light above can be replaced with a different wavelength of light that is absorbed by the nanoparticle (e.g., corresponding to an optical absorption peak or a plasmon resonance frequency of the nanoparticle). Thus, in some cases, the Diels-Alder cyclo-addition product does not absorb more than 3%, more than 5%, more than 10%, or more than 15% of incident photons having a wavelength within 30 nm, within 20 nm, or within 10 nm of the NIR optical absorbance peak of the nanoparticle.

For magnetic nanoparticles, heating can be achieved by application of an alternating magnetic field. As described above, this magnetic energy can be converted to thermal energy, which heats the magnetic particles. In some such embodiments, the applied alternating magnetic field can have a frequency ranging from 100 to 1,000 kHz, 150 to 950 kHz, 200 to 900 kHz, 250 to 850 kHz, 300 to 800 kHz, 350 to 750 kHz, 400 to 700 kHz, 450 to 650 kHz, or 500 to 600 kHz. Not intending to be bound by theory, it is believed that these alternating magnetic field frequencies are sub-toxic.

One observed benefit of using an alternating magnetic field to heat magnetic nanoparticles, compared to irradiating plasmonic metal nanoparticles with light that matches a resonant frequency of at least one of the nanoparticles is the ability to heat the nanoparticles at greater depths in a biological compartment. Without wishing to be bound by any particular theory, this result is believed to occur because the alternating magnetic field is not attenuated to the same degree as light is by the biological compartment (due to the presence of water or one or more biomolecules). Thus, in some cases, compositions and methods described herein can be particularly suitable for use in deep-tissue cancer imaging and/or therapy.

### III. Methods of Inducing Tissue Regeneration

In another aspect, methods of inducing tissue regeneration are described herein. In some instances, such a method comprises disposing a composition described herein in an environment, such as a biological compartment, and decomposing the Diels-Alder cycloaddition product, thereby severing the linker and decoupling the therapeutic species, as previously described in Section II. Any nanoparticle and linker described hereinabove in Section I can be used. In some embodiments, the therapeutic species comprises a tissue regenerative species, such as an osteogenic modulator, a chondrogenic modulator, an endotheliologenic modulator, or a myogenic modulator. Exemplary embodiments of tissue regenerative species comprise a nucleic acid, a microRNA (“miRNA”), a small interfering RNA (siRNA), a peptide, a small molecule, an antibiotic, an antifungal, an antibody, a protein, or any combination thereof. Specific embodiments of an osteogenic modulator, a chondrogenic modulator, an endotheliologenic modulator, a myogenic modulator can comprise one or more of the examples described in Section I herein.

In one embodiment, the tissue regenerative species is a miRNA-148b mimic. MiRN-148b induces differentiation of human autologous adipose derived mesenchymal stromal/stem cells (hASCs) into an osteogenic linage. In some instances, release of miRNA-148b into the biological compartment of a cell (such as hASCs) increases alkaline phosphatase (ALP) activity in the cell membranes and calcification (mineralization) of the cell. ALP is one of the early protein enzymes expressed during osteogenesis and is displayed on the extracellular portion of the plasma membrane, resulting in increased local concentration of inorganic phosphate, a mineralization promoter, and decreased concentration of extracellular pyrophosphate, an inhibitor of mineral formation. In some instances, release of miRN-148b using the methods described herein, induces upregulation in the expression of mRNA for early, middle, and late stage osteogenic marks, such as ALP, RunX2, osteocalcin (OCN).

### IV. Methods of Treating Cancer

In another aspect, methods of treating cancer are described herein. In some instances, the method comprises disposing a composition described herein in an environment, such as a biological compartment, and decomposing the Diels-Alder cycloaddition product, thereby severing the linker and decoupling the therapeutic species, as previously described in Section II. Any nanoparticle and linker described hereinabove in Section I can be used. In some embodiments, the therapeutic species comprises an anti-cancer species according to any of the embodiments described in Section I herein. In one embodiment, the anti-cancer species is a miRN-148b mimic. miRN-148b has been shown to act as a tumor suppressor and promote carcinogenesis. While not intending to be bound by any theory, it is believed that miRN-148b acts as a tumor suppressor by targeting specific oncogenes in a wide variety of tissues, dramatically suppressing the growth of cancer cells, attributable to induction of apoptosis and cell-cycle arrest at S-phase. In some instances, release of miRN-148b using the methods described herein, treats cancers by suppressing the growth of cancer cells, or killing cancer cells.

In another preferred embodiment, miR34a is used as an anti-cancer species for treating glioblastoma.

Additional aspects of compositions and methods of this disclosure are further illustrated in the following non-limiting examples.

### Example 1

### Synthesis of Silver Nanoparticles

Colloidal silver nanoparticles (SNPs) were prepared as follows, yielding SNPs of approximately 75 nm in diameter size. At room temperature, 35 mL of each 125 mM silver nitrate (AgNO₃) and 61.5 mM formaldehyde (HCOH) were incrementally added 0.5 mL/min into a pre-made solution of 0.5 g NaOH, 0.31 g HPC, 330 mL deionized (DI) water (18.2 MX), and 5 mL Antifoam A. For purification, the nanoparticles were filtered via dialysis and freeze-dried under vacuum for 72 h before use. A stock solution of 200 ppm in DI water was later prepared for further chemical modification.

### Example 2

### Synthesis of Magnetically-Activated Nanoparticles

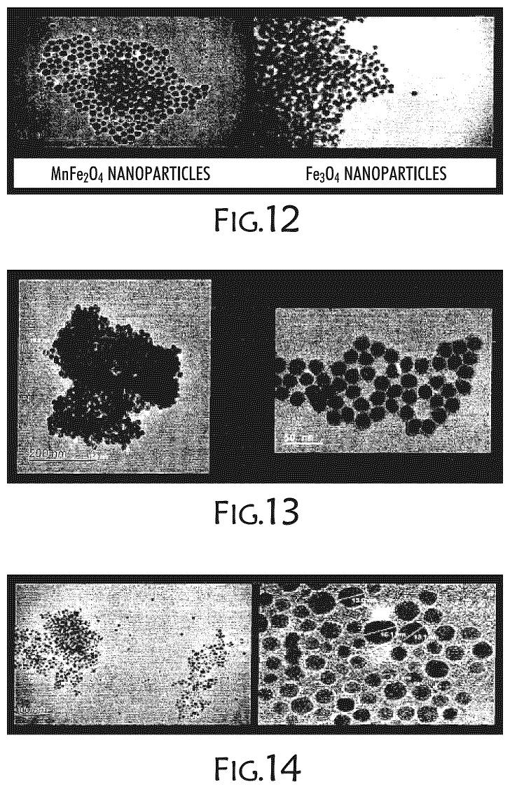

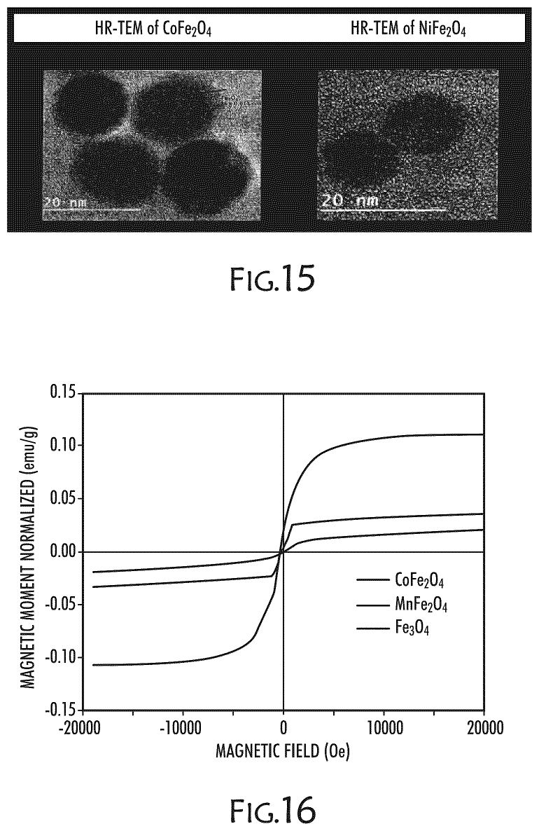

Magnetically-activated AuFe₃O₄ dumbbell nanoparticles were synthesized as described in, Yu, Heng, et al. “Dumbbell-like Bifunctional Au—Fe₃O₄ Nanoparticles.” Nano Letters 5.2 (2005): 379-82, which is incorporated by reference in its entirety herein. Under nitrogen flow, 0.30 ml Fe(CO)₅ (2 mmol) was injected into a pre-mixed solution consisting of 1.87 mL oleic acid (6 mmol), 1.97 mL oleylamine (6 mmol), 2.58 g 1,2-hexadecandiol (10 mmol) and 20 ml 1-octadecene at 120° C. for 20 min. After 3 min, 40 mg of HAuCl₄·(H₂O)₃ (0.1 mmol), 0.5 ml oleylamine (1.5 mmol) and 5 ml 1-octadecene were added to the solution. The mixture was then heated to ˜300° C. by reflux for 45 min, cooled to room temperature, and aerated for an hour. The particles were precipitated out with either iso-propanol or ethanol and a magnet bar, washed, and re-dispersed in hexane. FIGS. 12-15 show TEM images of MnFe₂O₄ and Fe₃O₄ nanoparticles (FIG. 12), CoFe₂O₄ nanoparticles (FIG. 13), NiFe₂O₄ nanoparticles (FIG. 14), and CoFe₂O₄ and NiFe₂O₄ nanoparticles (FIG. 15, HRTEM images) made in accordance with this procedure.

### Example 3

### Formation of Diels-Alder Linker

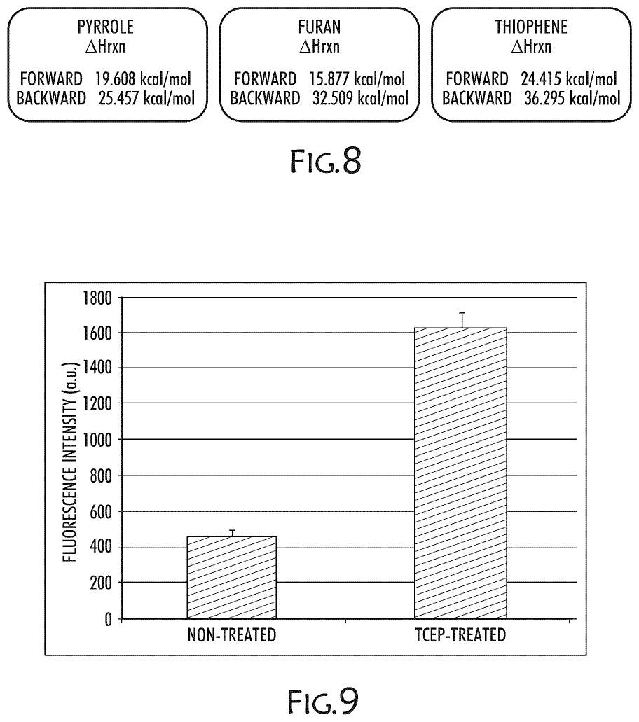

Three different linkers comprising a Diels Alder cycloaddition product were formed by the reaction shown in FIG. 5, where X is O (furan), S (thiophene), or NH (pyyrole). FIG. 8 includes the calculated forward and reverse ΔHᵣₓₙ for formation of the three different linkers by the (forward) Diels-Alder reaction in FIG. 5 and reverse (retro) Diels-Alder reaction in FIG. 6. The Diels-Alder cycloaddition product of the linker can be chosen, as was done here, such that the forward cycloaddition reaction is more energetically favorable than the backward (retro or reverse) reaction. This allows for the Diels-Alder cycloaddition product to be formed at subcleavage (or retro or reverse Diels-Alder reaction) temperatures.

For the cycloaddition between 6-maleimidohexanoic acid and 2-furanmethanethiol, 4.18 g of the dienophile, the maleimide, was combined with 1 mL of the diene reagent, in a 1:1 (v/v) dichloromethane/methanol (“DCM:MeOH”) solvent mixture. The reaction was allowed to proceed for 7 days under agitating conditions at room temperature in a sealed container. For the Diels-Alder reactions between the 2-thienylmethanethiol (0.5 mL) and the 6-maleimidohexanoic acid (2.11 g); and the pyrrole-2-carboxylic acid (0.555 g) and 6-maleimidohexanoic acid (2.11 g), the reagents were again mixed together in MeOH-only solvent. Both the reactions for the pyrrole and thiophene were carried out in an oil bath at 60° C. for 3 days under controlled ventilation. The bicyclic products between the different dienes and dienophile were purified by HPLC and characterized via MALDI-MS, ¹H and ¹³C NMR.MALDI, FTIR, 1H NMR, and ¹³C NMR confirm synthesis of all three linkers comprising pyrrole, furan, and thiophene cycloaddition product. Results for the linker comprising the furan cycloaddition product, the thiophene cycloaddition product and the pyrrole cycloaddition product are presented in Table 3 below. The ¹³C NMR peaks, the FTIR major peaks, and the M/Z ratio from the MALDI data all indicate formation of the respective linkers. The M/Z ratio (found) from the MALDI data indicates a product of the expected m/z ratio (calculated) in positive ion mode of the respective linkers.

TABLE 3

| | |
|---|---|
| 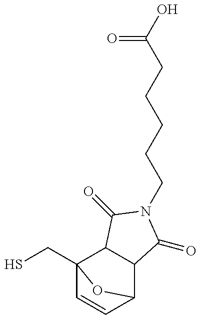 [SMILES: O=C(O)CCCCCN1C(=O)C2C3C=CC(CS)(O3)C2C1=O] | Purified with HPLC C18 Column; 100% H₂O flow at 0.3 mL/minYield: 21.6%¹H NMR (500 MHz, DMSO-d6) δ 1.24 (m, 2H), 1.46 (p, J = 7.5 Hz, 4H), 2.18 (t, J = 6.7 Hz, 2H), 2.92 (d, J = 6.4 Hz, 1H), 3.17 (m, 1H), 3.58 (m, 2H), 4.06 (m, 2H), 5.25 (t, J = 7.1 Hz, 1H), 6.37 (m, 1H), 6.51 (m, 1H). ¹³C NMR (500 MHz, DMSO-d6): δ 25.67, 27.38, 28.36, 33.26, 34.83, 39.62, 50.81, 51.11, 80.77, 92.88, 135.46, 139.69, 176.94, 177.24, 177.49MS (ESI+): m/z calculated for C₁₅H₂₁NO₅S, 325.40; found (M + H⁺) 326.1061. |
| Furan Diels- | |
| Alder linker | |
| 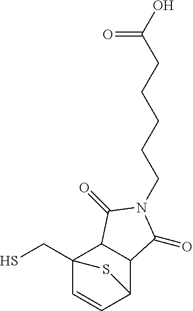 [SMILES: O=C(O)CCCCCN1C(=O)C2C3C=CC(CS)(S3)C2C1=O] | Purified with HPLC C18 Column; 1.3% ACN 98.7% H₂O flow at 0.3 ml/minute Yield: 24.8% ¹H NMR (500 MHz, DMSO-d6) δ 1.20 (m, 2H), 1.46 (m, 4H), 2.17 (t, J = 7.8 Hz, 2H), 2.64 (dd, J = 19.9, 4.3 Hz, 1H), 3.10 (dd, J = 20.1, 9.5 Hz, 1H), 3.31 (m, 2H), 3.42 (dd, J = 12.6, 7.36 Hz, 2H), 3.87 (dd, J = 9.5, 4.4 Hz, 1H), 6.74 (m, 1H), 6.91 (m, 1H). ¹³C NMR (500 MHz, DMSO-d6): δ 24.49, 26.15, 27.21, 33.50, 35.69, 39.42, 47.71, 51.21, 51.68, 52.58, 134.89, 140.64, 175.54, 175.55, 177.02MS (ESI+): m/z calculated for C₁₅H₂₁NO₄S₂, 341.44; found (M + H⁺) 342.08. |
| Thiophene | |
| Diels-Alder linker | |
| 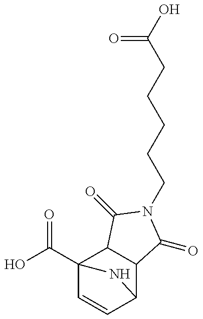 [SMILES: O=C(O)CCCCCN1C(=O)C2C3C=CC(C(=O)O)(N3)C2C1=O] | Purified with HPLC C18 Column; 20% ACN and 80% H₂O flow at 0.3 ml/minute. Yield: 23.8%¹H NMR (500 MHz, DMSO-d6) δ 1.22 (m, 2H), 1.55 (m, 4H), 2.18 (t, J = 7.4 Hz, 2H), 3.01 (m, 1H), 3.59 (m, 2H), 5.90 (dt, J = 17.7, 5.8 Hz, 1H), 6.13 (q, J = 2.7 Hz, 1H), 6.68 (m, 1H), 7.01 (m, 1H). ¹³C NMR (500 MHz, DMSO-d6) δ 24.45, 26.10, 27.20, 33.92, 38.43, 44.11, 51.67, 55.82, 56.11, 123.83, 134.93, 162.35, 171.57, 176.14, 177.25.MS (ESI+): m/z calculated for C₁₅H₁₈N₂O, 322.23; found (M + H⁺): 323.16. |
| Pyrrole | |
| Diels-Alder linker | |

### Example 4

### Connection of Nanoparticle to Linker

For nanoparticle attachment to any one of the linkers described in Example 3, the solutions described in Examples 1 and 2 were dried with nitrogen gas to remove excess solvent and concentrate the samples prior to suspension, without HPLC purification. Additionally, in the case of the pyrrole-2-carboxylic acid Diels-Alder reaction, the diene was first cross-linked with cysteamine using EDC coupling chemistry for SNP modification. Briefly, EDC (1-Ethyl-3-(3-dimethylaminopropyl)-carbodiimide) (0.500 g), NHS (N-hydroxysuccinimide) (0.800 g) and cysteamine (0.400 g) were added to the pyrrole-2-carboxylic acid (0.555 g) and agitated overnight at room temperature. Three 1 mL-aliquoted SNPs was centrifuged (10,000 rpm, 15 min) and after removal of the supernatant, 0.5 mL of each Diels-Alder product was added directly to one of the 1 mL aliquots of the pelleted SNPs. The SNP surface modification step was left to proceed at room temperature for 24 h for all the three generated products. The nanoparticles were then washed three times by centrifuging for 10 min at 10,000 rpm consecutively, in which each step involved removal of the supernatant and resuspension in 1 mL of 70% (v/v)ethanol.

### Example 5

### Connection of Nanoparticle Linker with Fluorescent-Labeled Small Interfering RNA (si-RNA)

The above-described three linkers (pyrrole, furan, and thiophene-based) were respectively reacted with EDC/NHS (N-ethyl-N′-(3-(dimethylamino) propyl)carbodiimide/N-hydroxysuccinimide) and a fluorescently-labeled si-RNA. In this embodiment, a 3′amine/5′-FAM modified anti-sense RFP single stranded siRNA was used having a sequence of UUGGAGCCGUACUGGAACUUG (“miRN-148b”). Specifically, to conjugate a FAM-tagged RFP antisense siRNA mimic, an EDC coupling protocol was used with 100 mL of an aqueous EDC/NHS (100 mM) stock solution added to each of the three resuspended nanoparticle aliquots prepared in Example 3, followed by 50 mL of the amine-terminated siRNA (4 mM). After 24 h, the particles were again centrifuged, washed, and resuspended in DI water. A control sample in which cysteamine modified SNPs were linked to the 6-maleimidohexanoic acid via EDC coupling was also prepared, similarly to the pyrrole-based reaction described above, but without addition of the diene, to test the stability of both the amide and thiol linkages. Conjugation of the linkers and siRNA was tested by chemically reducing the Ag-linker bond using TCEP reagent (tris(2-carboxyethyl)phosphine hydrochloride) and measuring FAM intensity of the supernatant. The products of these reactions (pyrrole, furan, and thiophene-based) were analyzed. For example, the temperature dependency of the retro Diels Alder reaction (and release of the fluorescent si-RNA) was evaluated.

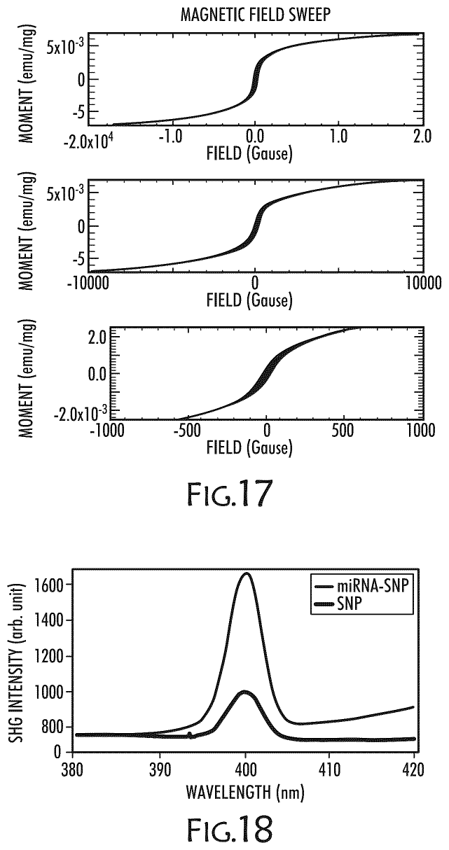

FIG. 9 shows fluorescence intensity of a control (non-treated) sample and a chemically cleaved (treated) sample including a product formed by reacting the furan-based linker with EDC/NHS and a fluorescently-labeled si-RNA. The fluorescence intensity of the treated sample is assumed to indicate maximum release (due to cleavage/retro (or reverse) Diels Alder reaction). The magnetic moment of three different nanoparticles used were also measured. FIG. 16 shows hysteresis loops for each of these nanoparticles, and FIG. 17 shows a magnetic field sweep. Specifically for FIG. 16, the curve with smallest vertical change in y-direction is the MnFe₂O₄, the curve with the largest vertical change is the CoFe₂O₄, and the middle curve is Fe₂O₄.

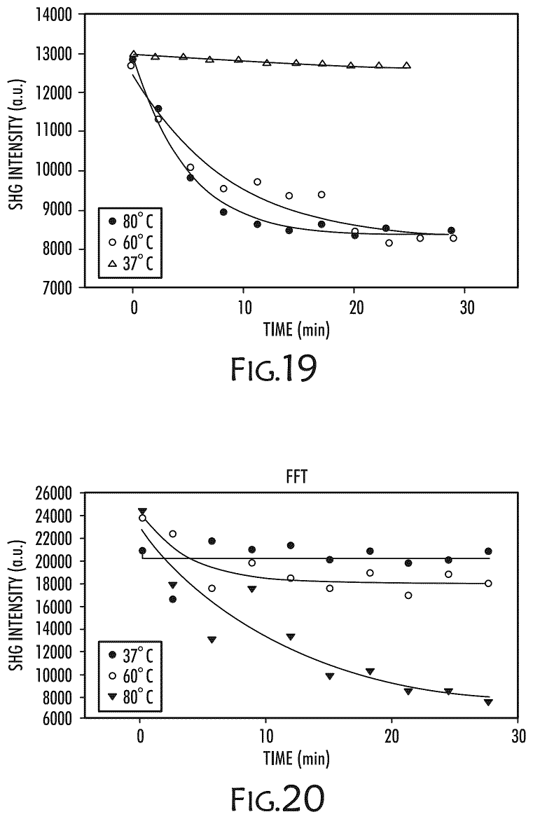

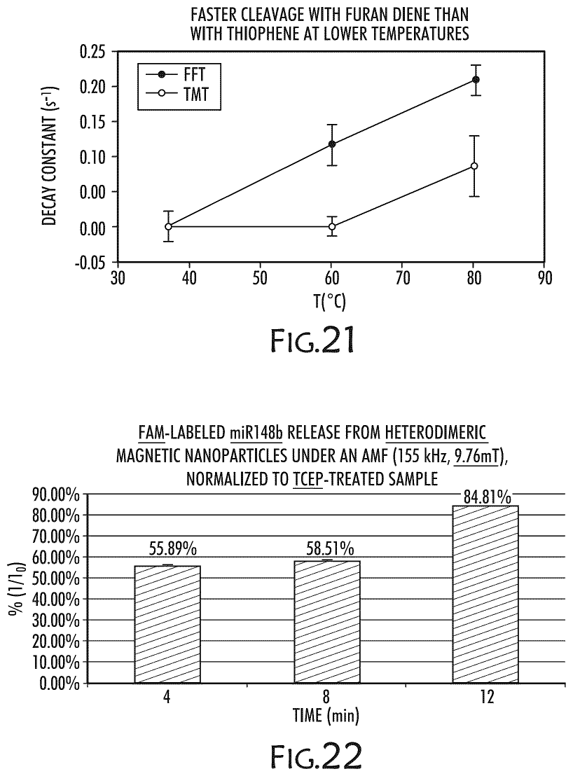

FIG. 18 graphically illustrates second harmonic intensity of a thiophene, furan, or pyrrole-based linker connecting an si-RNA to a plasmonic silver nanoparticle (SNP). FIG. 19 shows second harmonic (SHG) intensity, at particular temperatures, as a function of time for a composition comprising a siRNA-SNP with the pyrrole-based linker described herein in the Examples. Decreasing second harmonic intensity is indicative of cleaved via a retro (or reverse) Diels-Alder reaction, disconnecting or releasing SNP from siRNA. FIG. 20 shows second harmonic (SHG) intensity, at particular temperatures, as a function of time for a composition comprising a plasmonic silver nanoparticle connected to siRNA via the furan-based linker described herein in the Examples. Decay constants of compositions comprising a plasmonic silver nanoparticle connected to siRNA via the furan-based and thiophene-based linkers described herein in the Examples were measured and compared. FIG. 21 shows that decay constant for the composition comprising the furan-based linker was significantly higher than that for the composition comprising the thiophene-based linker at 60° C. and at 80° C. Compositions comprising heterodimeric Fe₃O₄/Au magnetic nanoparticles connected to fluorescently labeled microRNA (miRNA) were also formed. Relative release of the fluorescently labeled miRNA from the heterodimeric Fe₃O₄/Au was measured with respect to time at 155 kHz and 9.76 mT, normalized to a chemically released sample that was assumed to be near total release, as seen for example in FIG. 22.

### Example 6

### Anti-Cancer Effects of miRN-148b Released from Retro Diels-Alder Reaction in PAM212 Cells

The anti-cancer effects towards PAM212 skin cancer cells of miRN-148b released from compositions described herein were explored using a furan-based linker conjugated with a FAM-tagged miRN-148b and an SNP. The furan-based linker composition was prepared according to Examples 1-5. SNPs surface-modified with miRNA mimics via Diels-Alder linkage, were sterilized in 70/30% ethanol and water solution prior to washing and resuspension in RNAse-free water. Subsequently, appropriate nanoparticle solution volumes were added, based on required dosages, to the adherent monolayer PAM212 cells at optimal seeding densities, immersed in appropriate cell culture media. Transfection was allowed to occur at 37° C. for 24 h.

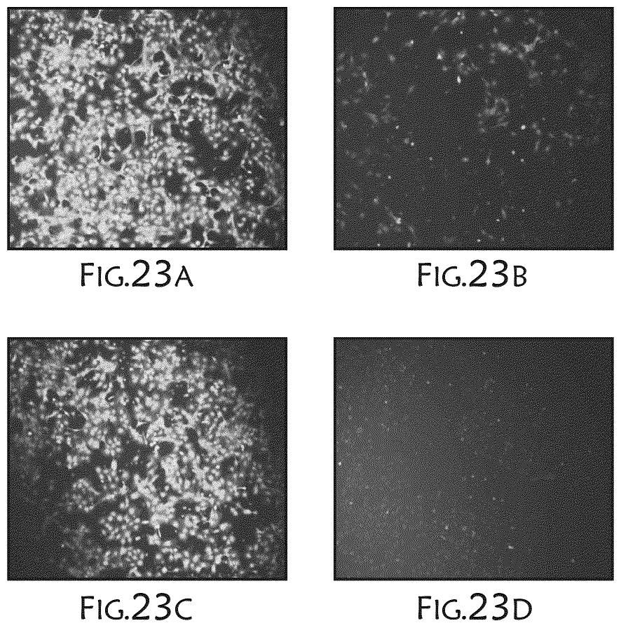

As seen in FIGS. 23A-23D, PAM212 cells were treated with the furan-based linker composition. FIG. 23A is a negative control showing fluorescence of the PAM212 cells in the presence of silver nanoparticles prepared in accordance with Example 1 after 7 days. FIG. 23B is a positive control showing PAM212 cells transfected with FAM-tagged miRN-148b 7 days after transfection. FIG. 23C shows PAM212 cells in the presence of a furan-based linker conjugated with a FAM-tagged miRN-148b and an SNP composition after 7 days, where the composition has not been light activated. FIG. 23D shows PAM212 cells in the presence of a furan-based linker conjugated with a FAM-tagged miRN-148b and an SNP composition after 7 days, where the composition has been light activated at 420 nm. As seen in FIGS. 23B and 23D, the PAM212 cells displayed reduced cell proliferation and growth in the presence of the positive control (FIG. 23B) and the light activated composition (FIG. 23D). Particularly, cell proliferation and growth was most dramatically reduced in the presence of the light activated composition in FIG. 23D. Cell growth in the negative control (FIG. 23A) and the composition not light activated (FIG. 23C) displayed normal cell behavior. Thus, light or magnetic activated compositions described herein can in some instances release anti-cancer/tumor therapeutic agents in cells using a retro Diels-Alder mechanism, and the released therapeutic agents can display in vivo activity towards cancer cells.

### Example 7

### Anti-Cancer Effects of miRN-148b Released from Retro Diels-Alder Reaction in Ras-Induced Keratinocyte Cells

The anti-cancer effects towards Ras-induced keratinocyte lung cancer cells of miRN-148b released from compositions described herein were explored using a furan-based linker conjugated with a FAM-tagged miRN-148b and an SNP. The furan-based linker composition was prepared according to Examples 1-5. SNPs surface-modified with miRNA mimics via Diels-Alder linkage, were sterilized in 70/30% ethanol and water solution prior to washing and resuspension in RNAse-free water. Subsequently, appropriate nanoparticle solution volumes were added, based on required dosages, to the adherent monolayer PAM212 cells at optimal seeding densities, immersed in appropriate cell culture media. Transfection was allowed to occur at 37° C. for 24 h.

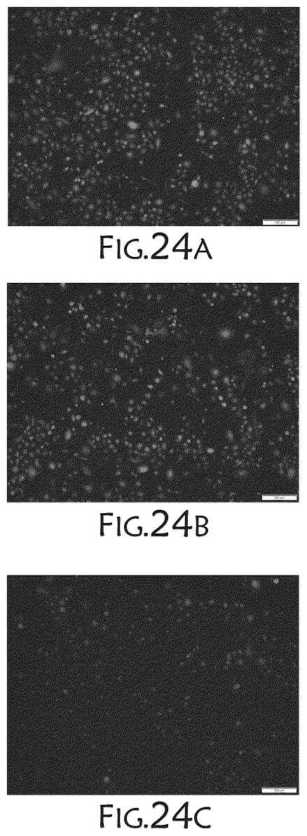

FIG. 24A is a negative control of keratinocyte cells in the absence of the composition and FAM-tagged miRN-148b after 7 days. FIG. 24B is a positive control of keratinocyte cells after 7 days, which have been transfected with FAM-tagged miRN-148b (100 nM). FIG. 24C shows keratinocyte cells that have been transfected with a composition comprising the furan-based linker conjugated with a FAM-tagged miRN-148b and an SNP, where the cells have been activated with light at 420 nm.

As seen in FIG. 24C, the keratinocyte cells displayed reduced cell proliferation and growth in the presence of light activated composition after 7 days. Cell growth in the negative control (FIG. 24A) and cells transfected with only FAM-tagged miRN-148b (FIG. 24B) displayed normal cell behavior. Thus, light or magnetic activated compositions described herein can in some instances release anti-cancer/tumor therapeutic agents in cells using a retro Diels-Alder mechanism, and the released therapeutic agents can display in vivo activity towards cancer cells.

### Example 8

### Tissue Regenerative Effects of miR-148b Released from Retro Diels-Alder Reaction

The tissue regenerative effects of miR-148b released from compositions described herein were explored using a furan-based linker conjugated with a FAM-tagged miRN-148b and a magnetic nanoparticle. Specifically, the osteo-inductive effects of miR-148b on hASCs were explored using the furan-based linker composition prepared according to Examples 1-5.

Magnetic nanoparticles surface-modified with miRNA mimics via Diels-Alder linkage, were sterilized in 70%/30% ethanol and water solution prior to washing and resuspension in RNAse-free water. Subsequently, appropriate nanoparticle solution volumes were added, based on required dosages, to the adherent monolayer hASCs cells at optimal seeding densities, immersed in appropriate cell culture media. Transfection was allowed to occur at 37° C. for 24 h.

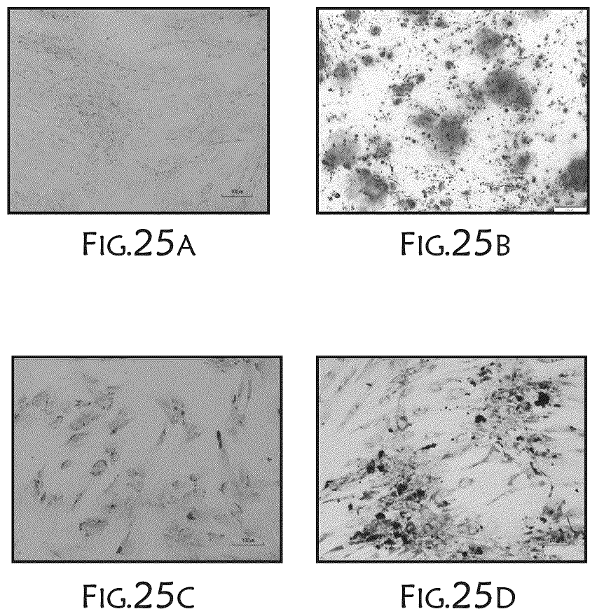

FIG. 25A shows a negative control of stromal media and Alizarin Red Staining D21 of hASCs cells. FIG. 25B shows a positive control of osteogenic media and Alizarin Red Staining D21 of hASCs cells. FIG. 25C shows a negative control of Alizarin Red Staining D21 of hASCs cells transfected with AuFe₃O₄ nanoparticles without RF activation. FIG. 25D shows a Alizarin Red Staining D21 of hASCs cells transfected with AuFe₃O₄ nanoparticles with miRNA148b attached with a furan-based Diels-Alder linker after radio frequency (RF) activation.

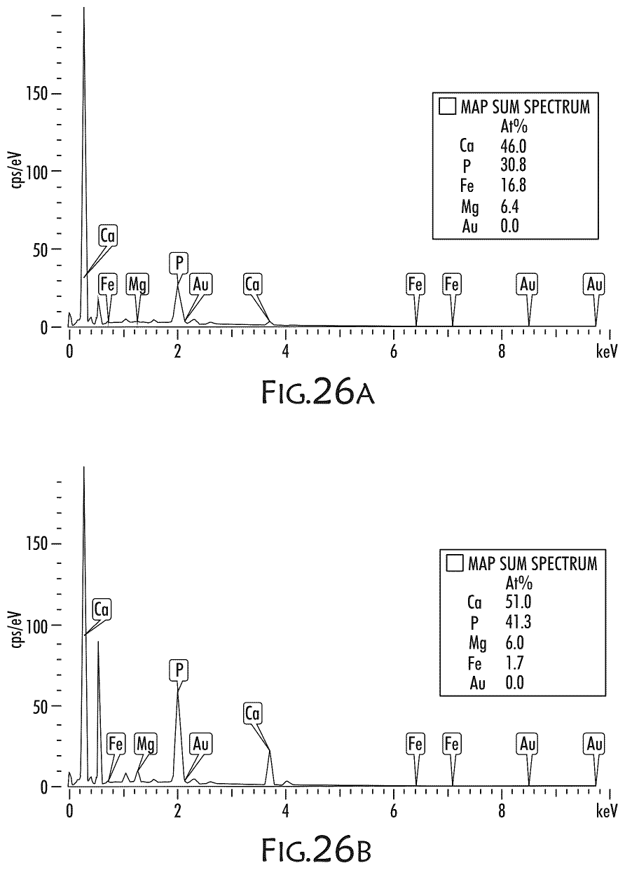

As seen in FIG. 25A, hASCs cells grown on stromal media display very low levels of calcification, whereas hASCs cells grown on osteogenic media display high levels of calcification (FIG. 25B). In the presence of AuFe₃O₄ nanoparticles without a linker and therapeutic agent, the hASCs cells display low levels of calcification (FIG. 25C). However, hASCs cells that have been transfected with AuFe₃O₄ nanoparticles with miRN-148b attached with a furan-based Diels-Alder linker, display increased calcification after RF activation, showing that the composition releases miRN-148b upon RF activation, and the miRN-148b induces osteogenesis (FIG. 25D). Thus, light or magnetic activated compositions described herein can in some instances release tissue regenerative therapeutic agents in cells using a retro Diels-Alder mechanism, and the released therapeutic agents can display in vivo tissue regenerative activity in the cells. Moreover, FIGS. 26A and 26B shows scanning electron microscope/energy dispersive x-ray spectroscopy (SEM/EDS) data on the mineralization effects in hASCs cells transfected with AuFe₃O₄ nanoparticles with miRN-148b attached with a furan-based Diels-Alder linker. FIG. 26A shows mineralization levels of hASC cells with RF Activated AuFe3O4 nanoparticles without miRN-148b. FIG. 26B shows mineralization levels of hASC cells after RF activation of AuFe₃O₄ nanoparticles with miRN-148b attached with a furan-based Diels-Alder linker. Comparison of FIG. 26A with FIG. 26B shows that after activation of the composition and release of the therapeutic agent, the hASC cells display increased levels of various minerals, such as calcium and phosphorus.

### Example 9

### Synthesis of Core-Shell-Shell Nanoparticles

### A. Synthesis of Gold Nanoparticle Seeds

Gold chloride (30 mL, 290 μM) was added to a 100 mL round-bottom flask equipped with a reflux condenser. The solution was vigorously stirred and heated until boiling. Sodium citrate (900 μL, 34 mM) was then added. After 10 min, the solution turned bright red and the reaction was cooled down to room temperature.

### B. Gold-Silver Core-Shell Nanoparticles

Gold nanoparticle seeds (200 μL of the previously prepared solution) were added to 10 mL of ultrapure water and vigorously stirred. Ascorbic acid (60 μL, 100 mM), silver nitrate (15 μL, 100 mM), and sodium hydroxide (75 μL, 100 mM) were then successively added. The obtained gold-silver core-shell nanoparticles were centrifuged and resuspended in 10 mL of ultrapure water.

### C. Gold-Silver-Gold Core-Shell-Shell Nanoparticles

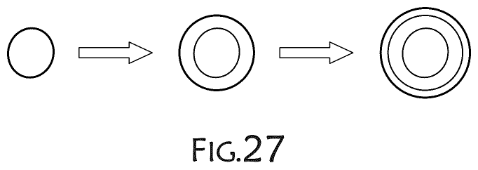

The previously prepared gold-silver core-shell nanoparticles were vigorously stirred. Gold chloride (100 μL, 29 mM), hydroquinone (100 μL, 0.03 M), and sodium citrate (25 μL, 34 mM) were then successively added. The obtained gold-silver-gold core-shell-shell nanoparticles were centrifuged and resuspended in ultrapure water prior to use (FIG. 27). With reference to FIG. 27, a gold core nanoparticle is schematically illustrated on the far left; a gold-silver core-shell nanoparticle is illustrated in the middle (with the outer sphere/circle representing a silver shell formed over the gold core); and a gold-silver-gold core-shell-shell nanoparticle is illustrated on the right (with the outermost sphere/circle representing a gold shell formed over the silver shell, which is itself formed over the gold core).

### D. Materials and Methods

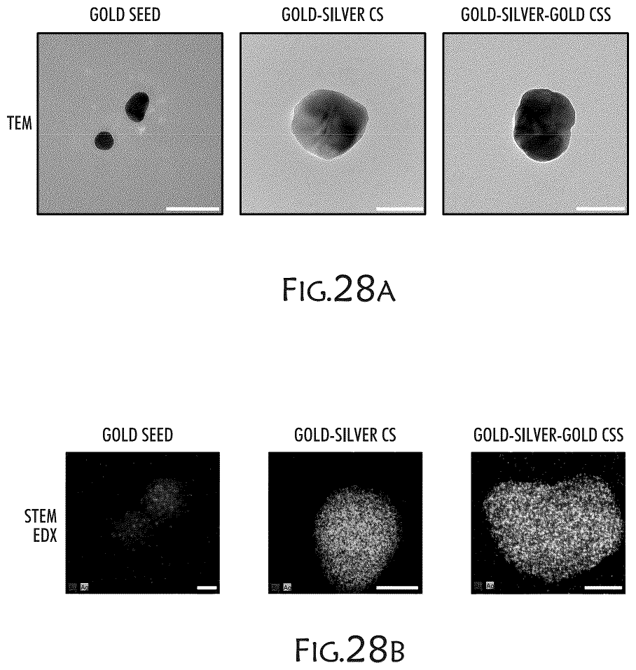

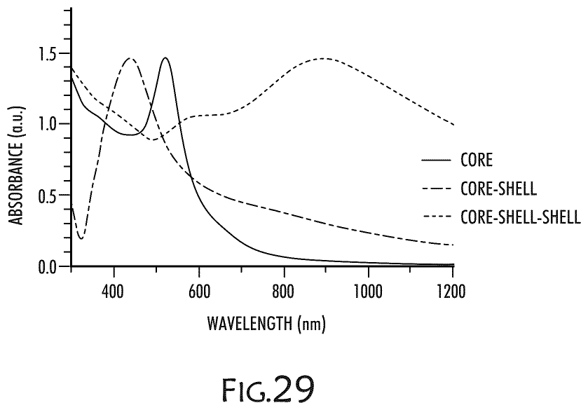

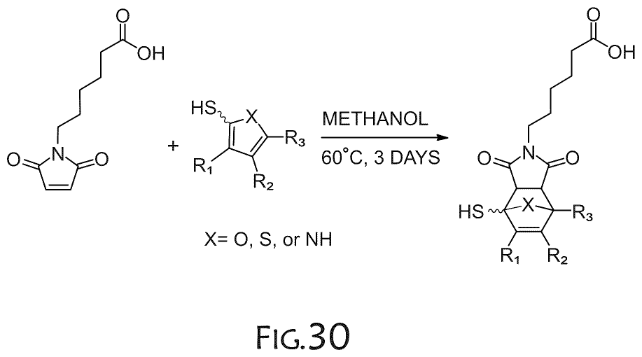

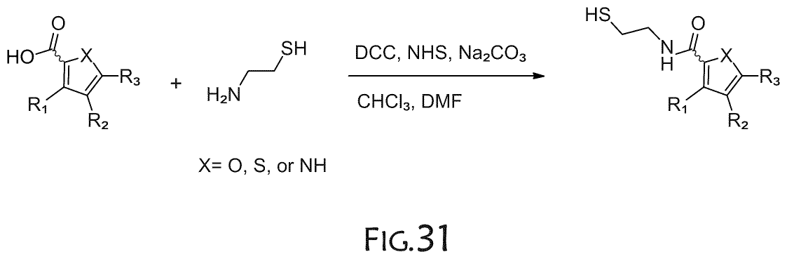

Gold (III) chloride trihydrate (HAuCl₄·3H₂O) was used for the synthesis of the nanoparticles. All stock solutions were prepared using ultrapure water. Washing of the nanoparticles by successive centrifugation and redispersion in ultrapure water was used to remove the excess reagents before the next synthesis step. To avoid spikes on the surface of the nanoparticles and obtain a smooth surface (FIG. 28), a very low concentration of potassium iodide (micromolar range) was added. Both the smoothness and the thickness of the shells can be adjusted to control the absorption peak of the core-shell-shell nanoparticles (FIG. 29). To obtain nanoparticles with a broad absorption peak in the near-IR range (over 800 nm), the surface of the shells is preferably smooth without spikes, and the thickness of the gold shell is at least 25 nm. Nanoparticles containing more than 2 alternating silver and gold shells may also be synthesized using these or similar protocols.

### Example 10

### Synthesis of Diels-Alder Linkers

### A. Example Protocol for the Synthesis of Pyrrole or Thiophene-Based Diels-Alder Linkers

Pyrrole or thiophene-based Diels-Alder linkers are typically prepared in a combination of methanol and dichloromethane (1:1 v/v) at 60° C. for 3 days. For example, using 2-thiophenemethanethiol as a diene and 6-maleimidohexanoic acid as a dienophile: 415 μL of 2-thiophenemethanethiol were combined with 7.5 mL of methanol and 7.5 mL of dichloromethane (DCM) in a glass vial. 6-maleimidohexanoic acid (1.75 g) was then added to the glass vial prior to closing it, wrapping it in aluminum foil and heating it at 60° C. for 3 days.

### B. Example Protocol for the Synthesis of Furan-Based Diels-Alder Linkers

Furan-based Diels-Alder linkers are typically prepared in a combination of methanol and dichloromethane (1:1 v/v) at room temperature for 7 days. For example, using 2-Furanmethanethiol as a diene and 6-maleimidohexanoic acid as a dienophile: 6-maleimidohexanoic acid (1.6 g) was dissolved in methanol (7.5 mL) and dichloromethane (7.5 mL) in a glass vial. 2-Furanmethanethiol (380 μL) was then added to the mixture. The vial was sealed, protected from light using aluminum foil, and the reaction allowed to proceed for 7 days at room temperature under agitation.

### Example 11

### Plasmonic Nanoparticle Functionalization with Linker and Therapeutic Species

In this Example, a sulfhydryl group on a Diels-Alder linker permits attachment of the linker on the surface of the core-shell-shell plasmonic particles described herein. One example protocol is as follows.

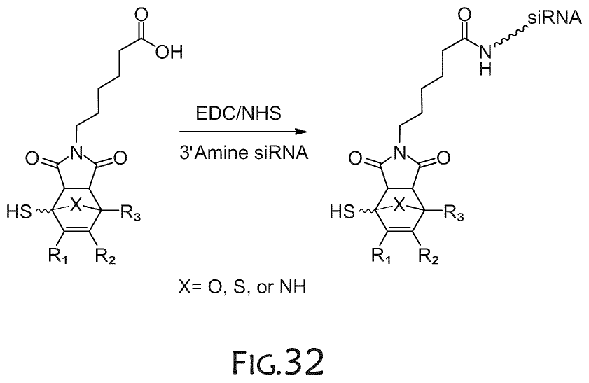

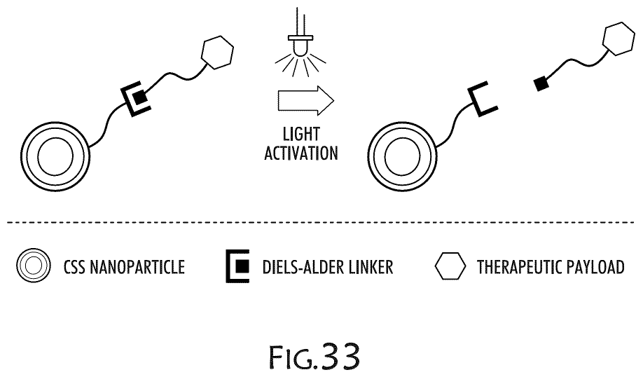

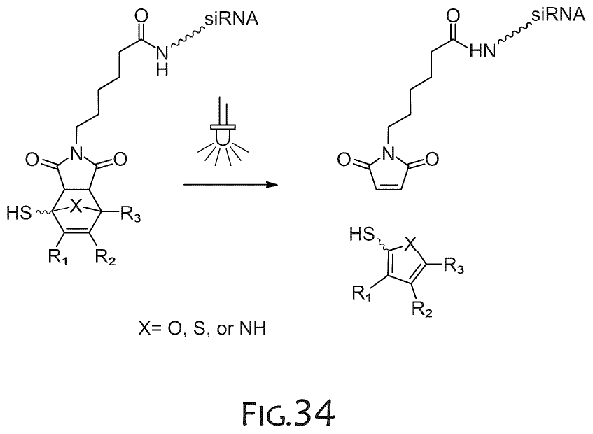

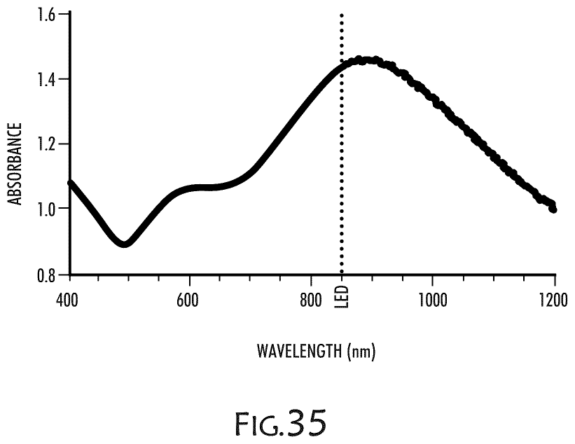

Nanoparticles from Example 9 were resuspended in a 1 mL aliquot of a previously prepared Diels-Alder linker. The nanoparticle surface modification step was left to proceed under agitation for 24 hours at room temperature. EDC/NHS coupling chemistry was used for the attachment of a siRNA to the Diels-Alder linker conjugated to the plasmonic particle (FIG. 32). Example protocol: for oligonucleotide addition, the nanoparticles were first centrifuged (10 000 g, 10 min) and resuspended in isopropanol thrice, followed by mixing with EDC/NHS (100 μL, 100 mM) and the respective 3′amine miRNA mimics (30 μL, 4 μM) for 24 hours at room temperature to allow for covalent coupling.

A cysteamine could be conjugated to a diene containing a carboxyl group to obtain a diene containing a sulfhydryl group (FIG. 31). The following is one example modification protocol of 4,5-Dimethyl-2-thiophenecarboxylic acid with cysteamine: 50 mg of 4,5-dimethyl-2-thiophenecarboxylic acid, 24.7 mg of cysteamine, 143.3 mg of DCC, 95.5 mg of NHS, and 477.6 mg of Na₂CO₃ were dissolved with 42 mL of DMF and 100 mL of chloroform in a 250 mL round-bottom flask. The mixture was stirred vigorously for 12 hours at room temperature. The sample was then dried down, redissolved in DCM and washed with 1M HCl three times for an acid-base extraction, with the organic layer containing the product. The organic layer was dried down and purified using column chromatography (hexane, DCM, acetic acid). The amount of 4,5-Dimethyl-N-(2-sulfanylethyl)-2-thiophenecarboxamide obtained was 20 mg.

### Example 12

### Light Activation and Payload Release

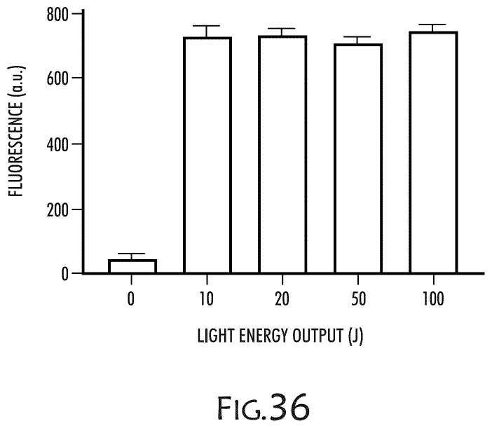

Plasmonic nanoparticles described herein generate heat via the photothermal effect after light irradiation, causing the Diels-Alder cycloadduct on the nanoparticle surface to undergo a retro reaction and releasing the conjugated siRNA payload (FIGS. 33, 34, and 35). In some cases, gold-silver-gold core-shell-shell nanoparticles produce a highly efficient step-function-like release profile (FIG. 36) when irradiated with as little as 10 J.

### Example 13

### Characterization and Use of Nanoparticle Compositions

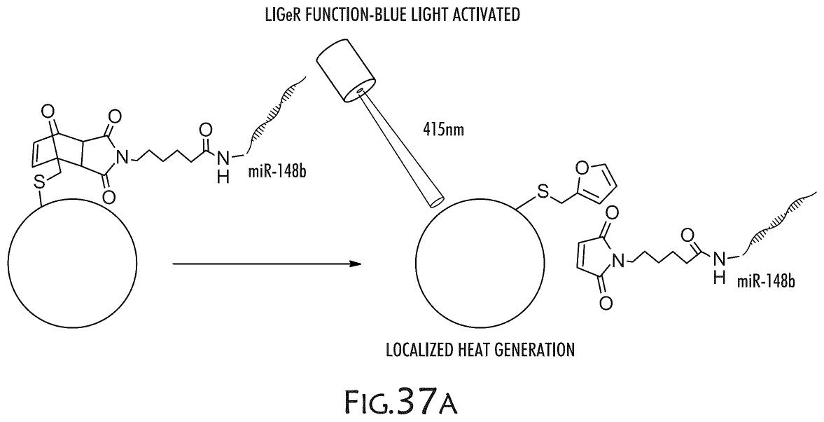

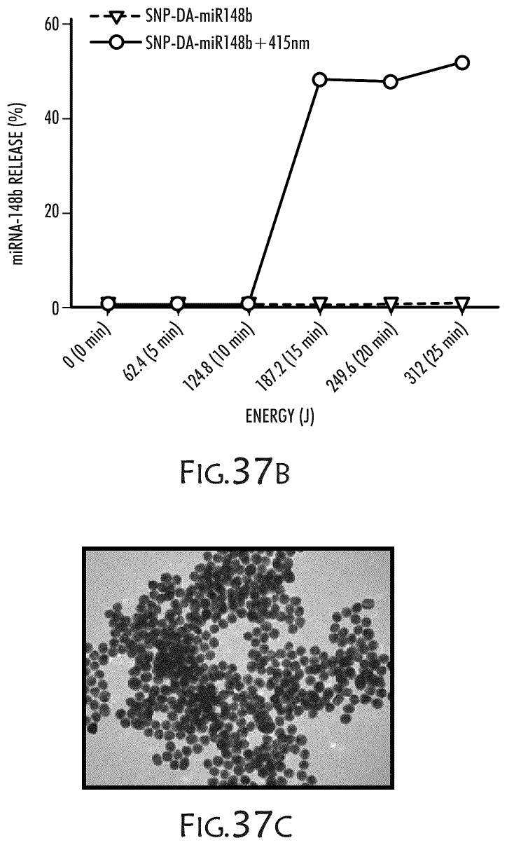

As illustrated in FIG. 37A, an example silver nanoparticle (SNP) was irradiated with 415 nm wavelength light producing localized heat generation, which provided energy for the retro Diels-Alder release of miR-148b. As illustrated in FIG. 37B, miR-148b was released over time upon exposure to energy from 415 nm wavelength light as exemplified in FIG. 37A. Light was initiated at time zero (0 min) and release of the therapeutic species tracked up to 25 min. A control under the same conditions, without 415 nm light, demonstrated that no miR-148b was released in the absence of energy from irradiation. A transmission electron microscope (TEM) image of the nanoparticles is illustrated in FIG. 37C.

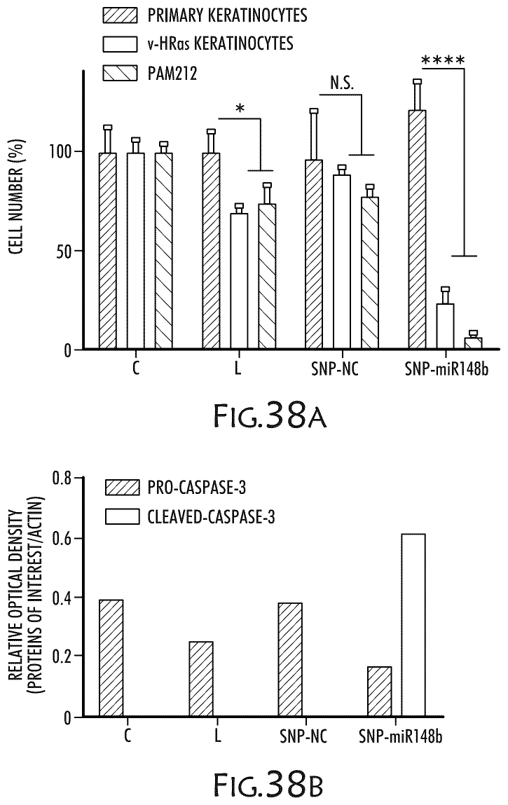

A comparative study was conducted using primary keratinocytes and keratinocytes having an HRas variant Ras gene (v-HRas), and PAM212 cells. These cells were cultured under different conditions and the total cell number tracked, as a percentage of initial cells. Results (FIG. 38A and FIG. 38B) showed that under control (C) conditions, the difference in cell number was not significant. Under exposure to light (L), the difference in cell number was slightly significant between the primary keratinocytes, and the PAM212 cells. Under exposure to a silver nanoparticle (SNP-NC) with scrambled non-coding siRNA, there was no significant (n.s.) difference in cell number. Under exposure to a silver nanoparticle loaded with miR-148b, there was a significant difference in cell number between primary non-cancerous keratinocytes, and the two other cancer cell lines. The relative DNA content of cells grown in containers was measured as a proxy for growth/survival. The two cancer cell lines showed a significant reduction in cell number compared to the primary non-cancerous cells. The mechanism of cell death was determined to be apoptosis, as the ratio of cleaved caspase-3 (the caspase enzyme in its active form) to pro-caspase-3 (the caspase enzyme before activation) increased significantly.

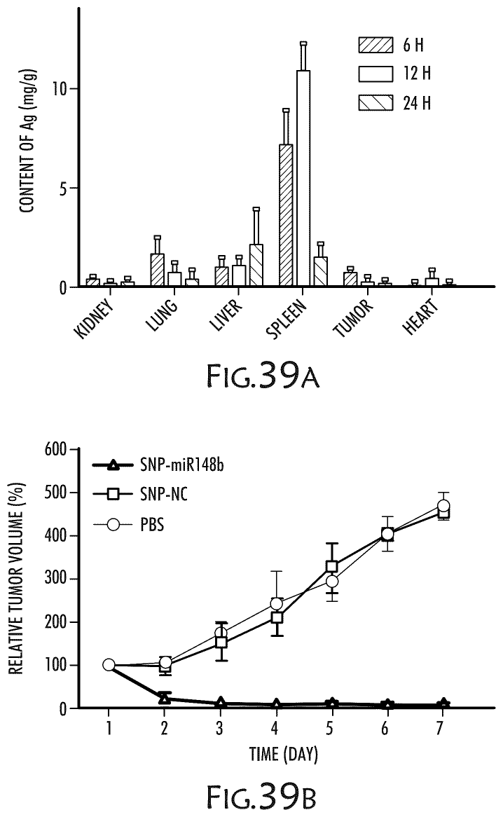

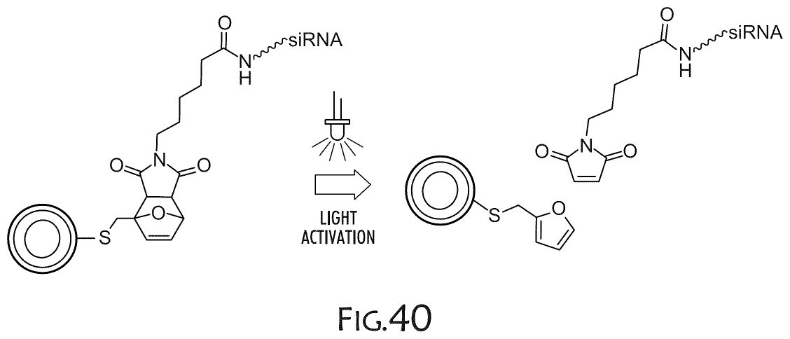

FIG. 39A shows the distribution, in mouse tissue, of the nanoparticles with miR-148b payload after retro-orbital intravenous injection at 6, 12 and 24 hours post injection. In kidney, lung and tumor tissue the concentration peaks at 6 hours post-injection, while the maximum concentration in kidney and spleen tissue is reached at 12 hours post-injections. Concentration in the liver continues to increase to 24 hours as the nanoparticle is cleared.

FIG. 39B displays the impact of different treatments on tumor volume for mice treated with the modalities: silver nanoparticles linked to miR-148b (SNP-148b), silver nanoparticles with a scrambled non-coding SiRNA (SNP-NC), or cell growth media (phosphate buffer saline, PBS). As shown, the PBS and nanoparticles with the non-coding siRNA display increases in volume that indicate tumor growth. However, the nanoparticle coupled to miR 148-b shows a reduction in tumor size which indicates that the tumor is almost completely gone after 7 days.

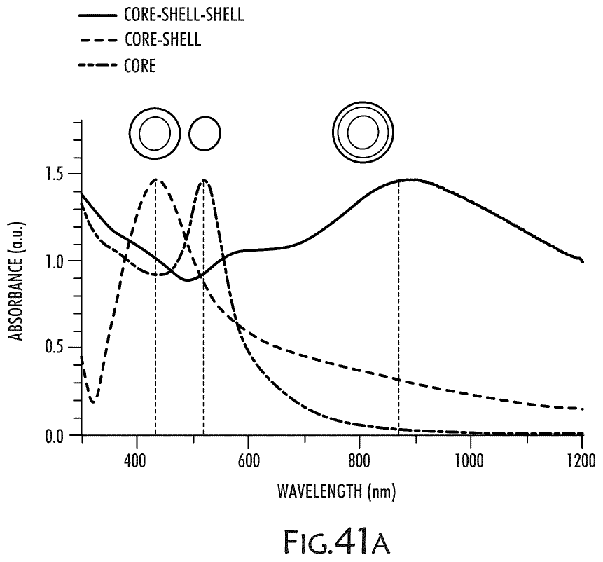

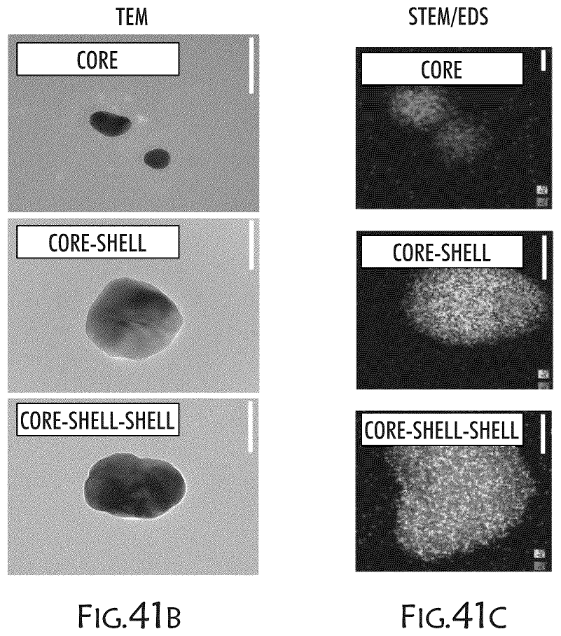

Gold-silver-gold core-shell-shell nanoparticles (prepared according to Example 9 above) were also used to provide beneficial clinical results as described herein. As illustrated schematically in FIG. 40, si-RNA is coupled to a nanoparticle via a Diels-Alder linker that is cleaved upon exposure to light which initiates a retro Diels-Alder reaction. FIG. 41A illustrates example absorbance spectra for core Au nanoparticles, Au—Ag core-shell nanoparticles, and Au—Ag—Au core-shell-shell (CSS) nanoparticles. The addition of shells shifts the localized surface plasmon resonance (LSPR) in a manner that is dependent on both the composition and the thickness of the shell material. The addition of a silver shell to a gold core shifts the LSPR from approximately 535 nm (green light) to approximately 420 nm (blue light). The addition of the further gold shell to the gold core-silver shell particle creates an Au—Ag—Au core-shell-shell particle and shifts the LSPR into the near IR region of the spectrum. Depending on the thickness of the final gold shell, the LPSR maximum can be shifted across a range of approximately 700 nm to approximately 1700 nm. This allows the LSPR of the particle to be tuned to discrete wavelengths in the nIR biological window. Moreover, as understood by one of ordinary skill in the art, the thickness of the final gold shell can be controlled based on gold precursor amount and/or gold shell growth conditions (e.g., time and temperature). FIG. 41B illustrates TEM images of the three nanoparticle types, and FIG. 41C illustrates scanning tunneling electron microscopy (STEM) and energy dispersive x-ray diffraction spectroscopy (EDS) images for the same nanoparticles, showing the gold (Au) and silver (Ag) content of the nanoparticles.

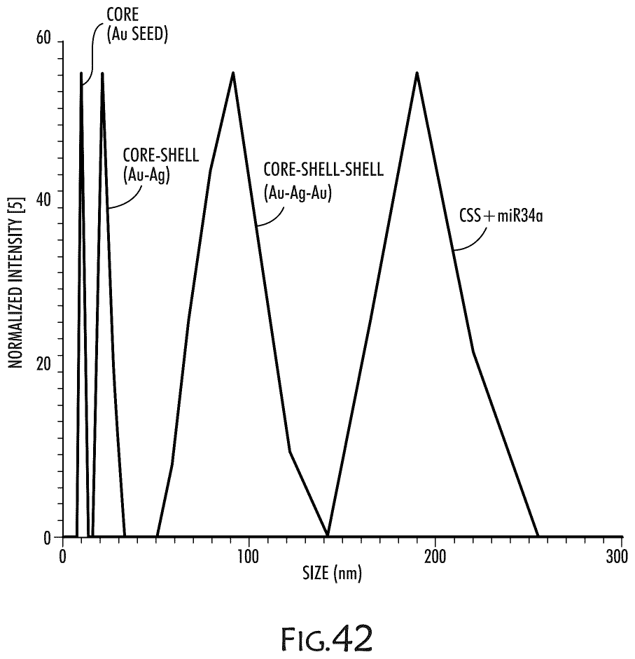

FIG. 42 illustrates a size histogram of the same three nanoparticle types: a gold core (core) NP, a gold-silver (Au—Ag) core-shell NP, a gold-silver-gold (Au—Ag—Au) core-shell-shell NP, and a core-shell-shell NP (CSS) attached to microRNA (CSS+miR34a). The Au—Ag—Au core-shell-shell NP size range of approximately 60 nm to 140 nm can provide efficient cellular transfection and a LSPR peak in the 700-900 nm near-infrared window I (NIR-I). Transfection efficiencies of up to 95% can be achieved with particles described above.

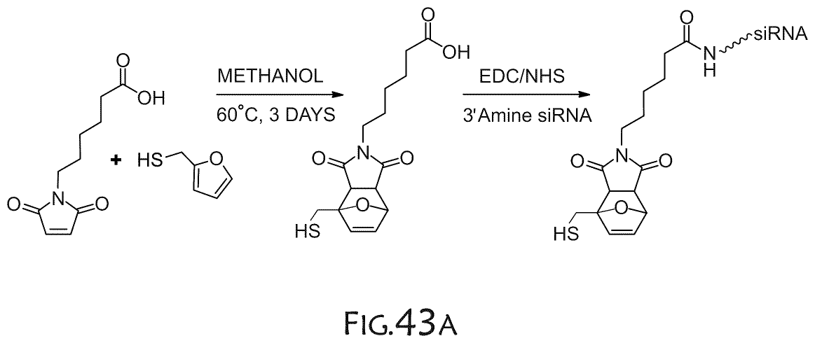

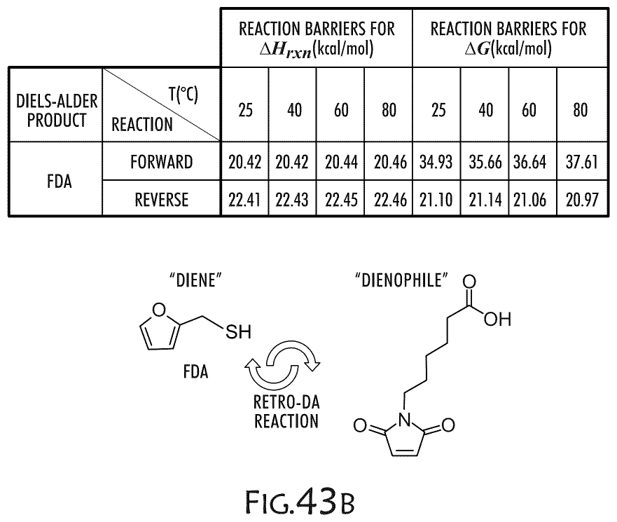

As described herein, various Diels-Alder linkers can be used to connect a nanoparticle described herein (such as a CSS nanoparticle) to a therapeutic species described herein (such as a miRNA species or combination of a plurality thereof). The synthesis of Diels-Alder linkers has been described extensively hereinabove. Additionally, FIG. 43A illustrates a reaction to produce a composition comprising a furan-based Diels-Alder linker. As illustrated in FIG. 43A, the synthetic process includes combining the diene and the dienophile in methanol and heating the composition at 80° C. for 3 days to produce an intermediate, which includes a Diels-Alder cycloaddition product. The intermediate (the Diels-Alder product) is then further reacted to couple the therapeutic species to the Diels-Alder product (where the therapeutic species is siRNA in this instance). In this instance, EDC/NHS chemistry is used to couple the carboxylic acid moiety of the intermediate to the siRNA via reaction with the 3′ amine of the siRNA.

As illustrated in FIG. 43B (and as described elsewhere herein), the reaction between a diene (e.g., a furan) and a dienophile can be reversible. The reaction barriers (enthalpy and Gibbs free energy) for the forward and reverse Diels-Alder reactions of the illustrated furan-based Diels-Alder linker (FDA) at different temperatures are shown in FIG. 43B.

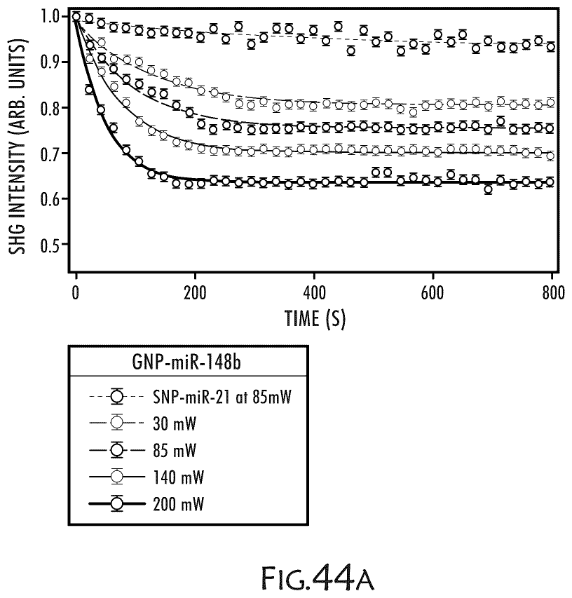

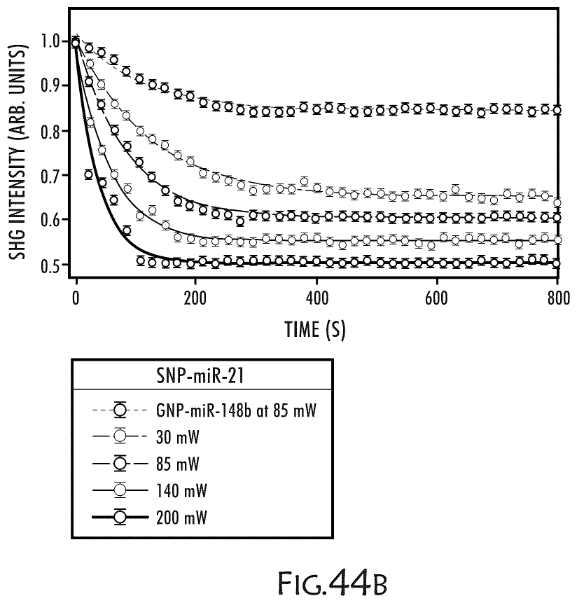

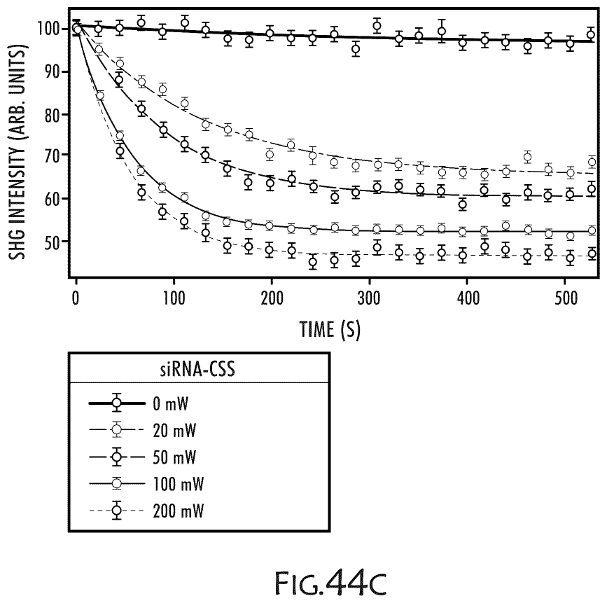

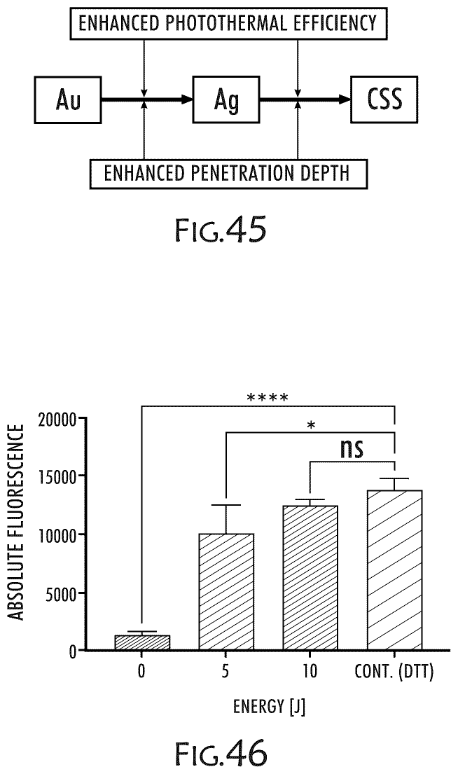

FIGS. 44A-C illustrate the relative photothermal efficiency (indicated by SHG intensity) of three particle types: a Au particle (FIG. 44A), a Ag particle (FIG. 44B), and a Au—Ag—Au core-shell-shell particle (FIG. 44C). The Au—Ag—Au core-shell-shell particle has significantly greater photothermal quantum efficiency than the core nanoparticles (particularly as compared to the gold particles). To obtain the data illustrated in FIGS. 44A-C, a titanium:sapphire oscillator laser centered at 800 nm with a 70 fs pulse duration, 80 MHz repetition rate, and 2.6 W average output power is used. The SHG signal is collected in the forward direction using a high-sensitivity charged-coupled device (CCD) spectroscopy detector coupled to the monochromator spectrograph. A portion of the oscillator laser beam passes through a beam splitter to seed the amplifier laser. The amplifier produces a laser beam centered at 800 nm with a 75 fs pulse duration, a 10 kHz repetition rate, and a 7 W average output power. The amplifier laser is used to irradiate the CSS nanoparticle sample to induce the photothermal cleaving of the siRNA, while the remaining portion of the oscillator laser is used to monitor the photothermal cleaving kinetics in real time using SHG spectroscopy. A computer-controlled beam block is used to block the amplifier irradiation beam while taking SHG signal with the oscillator laser. For the SHG measurements, the oscillator laser power is attenuated to a constant value of 300 mW to minimize photothermal release from the CSS surface while probing the kinetics. A 1 cm×1 cm quartz cuvette is used to contain 2 mL of the nanoparticle sample. The amplifier laser power is varied, and the photothermal cleaving kinetics are monitored as a function of irradiation time and power.

FIG. 46 illustrates the efficiency of the photo initiated retro Diels-Alder reaction and release of the fluorophore labelled siRNA as a function of applied energy. The retro Diels-Alder reaction was initiated with 5 joules (J) of 860 nm light, which resulted in approximately 70% of the siRNA payload release. Irradiating the particles with 10 J of 860 nm light can result in approximately 100% release of the fluorophore labelled siRNA. Treatment with the chemical releasing agent Dithiothreitol (DTT), is used as the standard to determine 100% release from the particle.

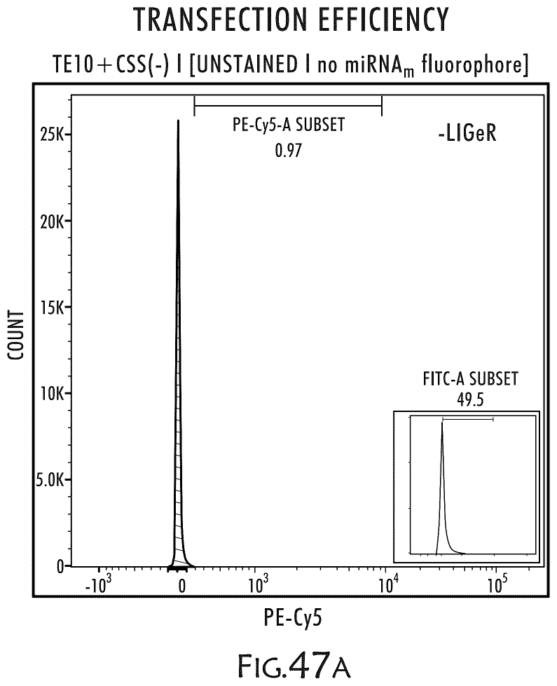

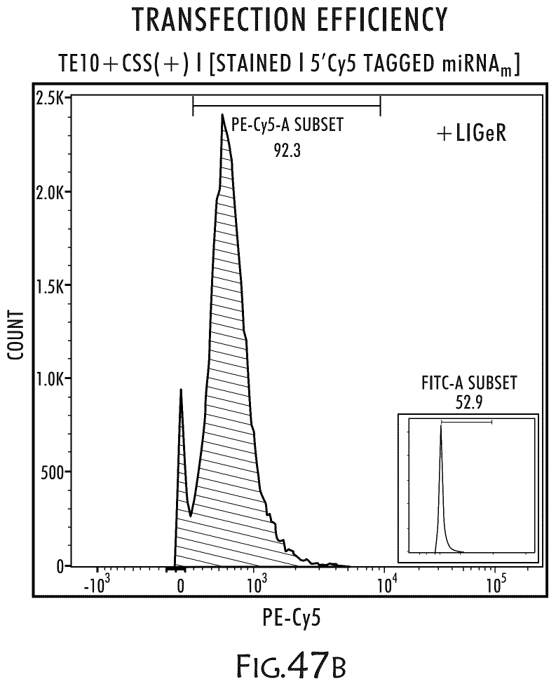

FIG. 47A illustrates the flow cytometry results from the no particle control for transfection efficiency in TE10 esophageal cancer cells.

FIG. 47B illustrates flow cytometry results of the uptake of Au—Ag—Au core-shell-shell nanoparticles with a Cy5 fluorophore labeled siRNA payload. When gated to the no particle control, a transfection efficiency of 92% is demonstrated.

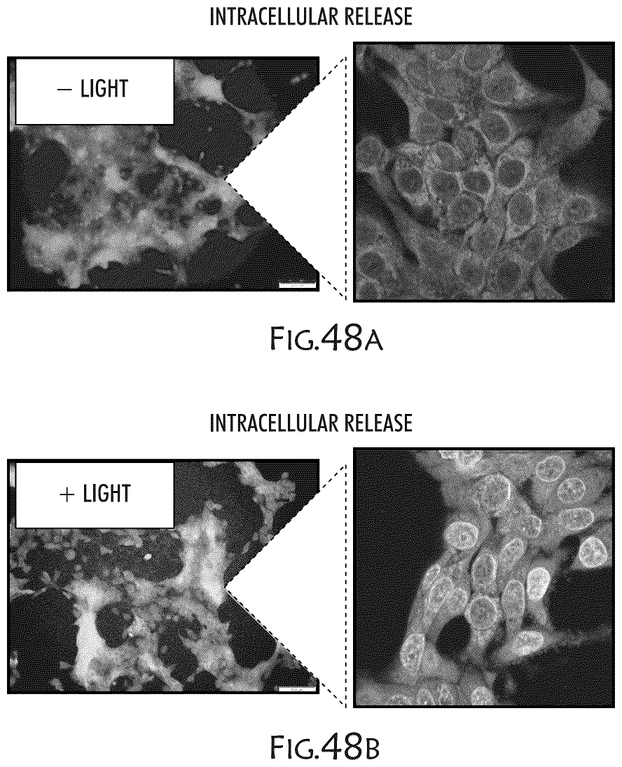

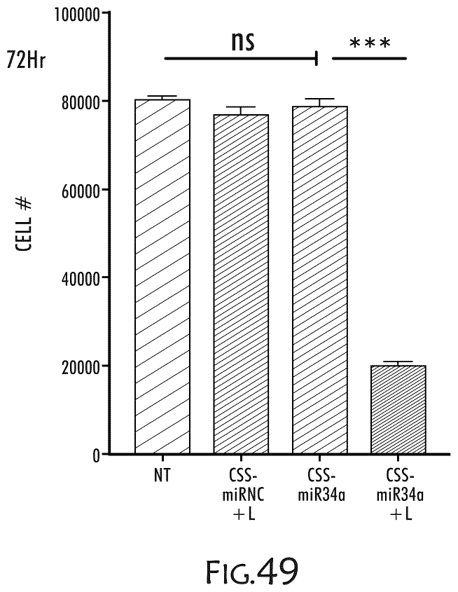

FIG. 48A illustrates the no light control for intracellular release experiments of TE10 cells transfected with Au—Ag—Au core-shell-shell nanoparticles with a Cy5 fluorophore labeled siRNA payload. The fluorophore is quenched when the siRNA is attached to the particle, so only the scattered light from the particle (red color, shown in grayscale here) and the nuclear staining (blue color, shown in grayscale here) is visible.

FIG. 48B illustrates the NIR light activated release of the Cy5 fluorophore labeled siRNA payload (green color, shown in grayscale here) from the Au—Ag—Au core-shell-shell nanoparticles, when nanoparticle transfected TE10 cells are irradiated with 860 nm light. After release of the siRNA, the Cy5 fluorophore is no longer quenched by proximity to the particle and the fluorescence becomes visible, indicating controlled release.

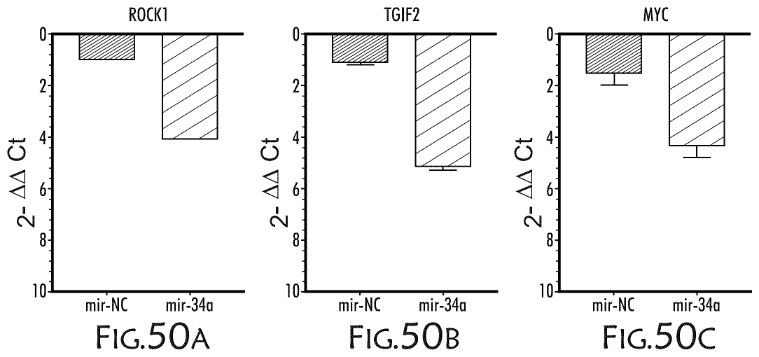

FIG. 49 illustrates a graph of cell number (#) on exposure to different conditions. NT indicates no treatment, CSS indicates a core-shell-shell nanoparticles, CSS-miR ##indicates a core-shell-shell nanoparticle linked to the micro-RNA listed, and ‘L’ indicates light is present (specifically light providing activation energy to heat the nanoparticle to initiate a retro Diels-Alder reaction). As illustrated, the greatest reduction in cell count is seen when cells are treated with CSS-miR34a+L.

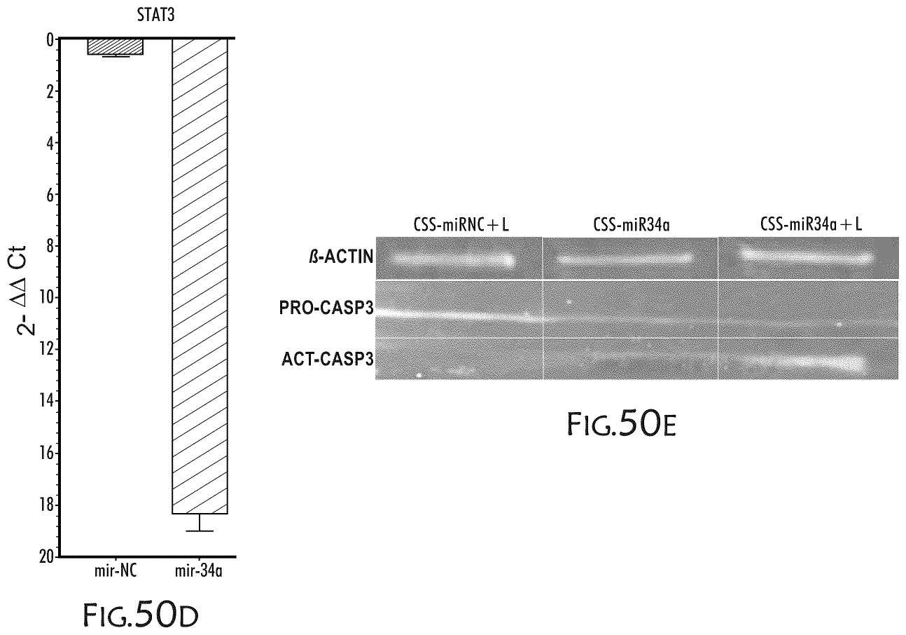

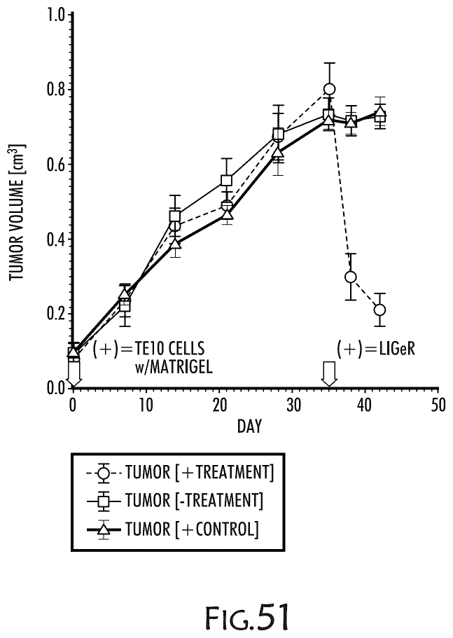

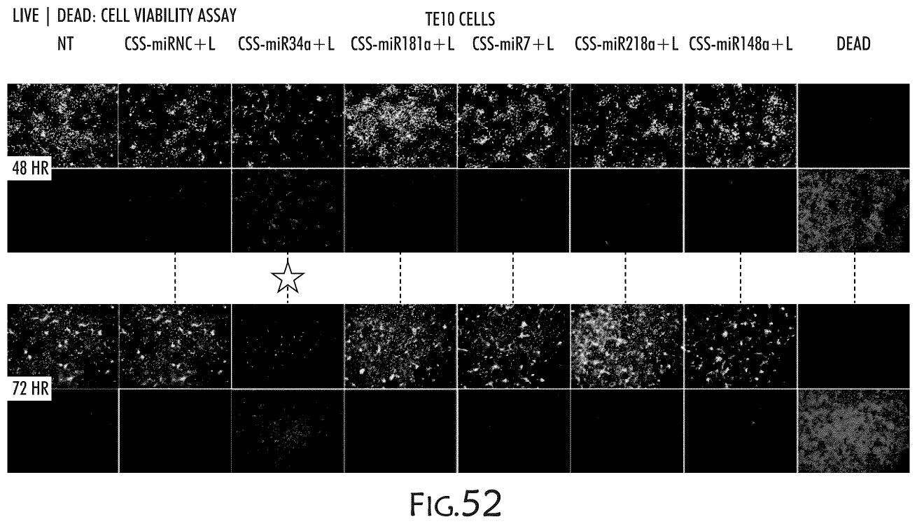

FIGS. 50A-49D illustrate graphs displaying the change in counts of measured tumor and/or cancer growth factors: ROCK1, TGF2, MYC, and STAT3. As illustrated in the figures, counts were measured under two conditions, in the absence of inhibitory microRNA (mir-NC) and in the presence of inhibitory microRNA (mir-34a). As shown, the decrease in count is greatest in the presence of inhibitory microRNA for each of the genes studied.

FIG. 50E displays an images of a gel stained to indicate the gene on the left under the conditions shown at the top.

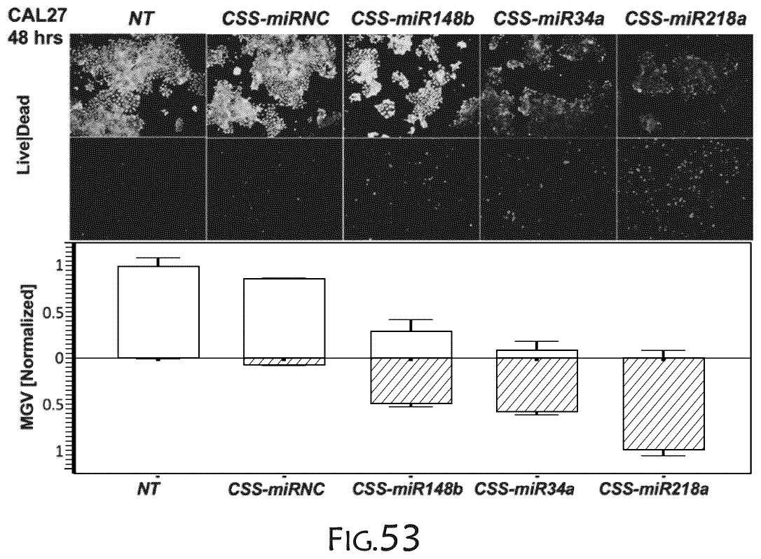

FIG. 51 illustrates a graph of tumor volume in mice provided different treatment modalities. In the control experiment, treatment is provided with no active agent. In the present case, a core-shell-shell nanoparticle was provided that was not attached to miRNA and/or siRNA. In the −Treatment experiment, no treatment is provided. As expected, the −Treatment and Control experiment display almost matching profiles indicating that tumor growth is unaffected in these experiments. In the +Treatment experiment, an example composition according to the present disclosure is provided, which includes a CSS nanoparticle and a therapeutic species linked to the CSS nanoparticle via a Diels-Alder cyclo-addition product.

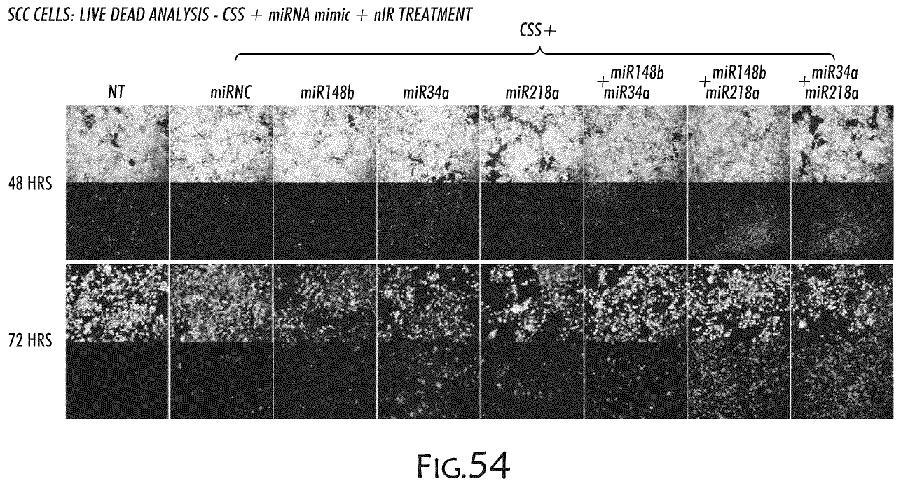

FIGS. 52-54 illustrate live/dead staining of TE10 cells (FIG. 52), Cal27 cells (FIG. 53), and SCC cells (FIG. 54) transfected with Au—Ag—Au core-shell-shell (CSS) particles with miRNA mimic payloads and light activated with 850 nm light to release miRNA mimics, according to some embodiments described herein. Cells were treated with either particles carrying only one miRNA mimic or with combinations of particles carrying different miRNA mimics.

Some additional, non-limiting example Embodiments are described below.

Embodiment 1. A composition comprising:
a core-shell-shell nanoparticle comprising a core, a first shell overlying the core, and a second shell overlying the first shell;
a therapeutic species; and
a linker joining the nanoparticle to the therapeutic species,
wherein the linker comprises a Diels-Alder cyclo-addition reaction product,
wherein the nanoparticle has an optical absorbance peak in the near infrared (NIR) region of the electromagnetic spectrum, and
wherein the Diels-Alder cyclo-addition reaction product does not have an optical absorbance peak within 100 nm of the optical absorbance peak of the nanoparticle.

Embodiment 2. The composition of Embodiment 1, wherein the core, the first shell, and/or the second shell of the nanoparticle is formed from a metal or an alloy, mixture, or combination of metals.

Embodiment 3. The composition of Embodiment 1, wherein the nanoparticle comprises a gold-silver-gold core-shell-shell nanoparticle.

Embodiment 4. The composition of any of the preceding Embodiments, wherein the second shell of the nanoparticle has an average thickness of at least 25 nm.

Embodiment 5. The composition of Embodiment 4, wherein the nanoparticle has a surface roughness of no greater than 10 nm.

Embodiment 6. The composition of Embodiment 5, wherein the exterior surface of the nanoparticle is free or substantially free of spikes or protrusions having a height of 2 nm or greater.

Embodiment 7. The composition of any of the preceding Embodiments, wherein the nanoparticle has an optical absorbance peak between 800 nm and 900 nm.

Embodiment 8. The composition of Embodiment 1, wherein:
the nanoparticle comprises a gold-silver-gold core-shell-shell nanoparticle;
the nanoparticle has a diameter or size of 60 nm to 140 nm; and
the nanoparticle has a localized surface plasmon resonance (LSPR) peak within a range of 700 nm to 900 nm.

Embodiment 9. The composition of any of the preceding Embodiments, wherein:
the Diels-Alder cyclo-addition reaction product has a forward reaction activation energy and a backward reaction activation energy, and
the backward reaction activation energy is at least 1.5 times the forward reaction activation energy.

Embodiment 10. The composition of any of the preceding Embodiments, wherein the backward reaction activation energy is greater than the thermal energy provided by aqueous surroundings of the nanoparticle at a temperature of 310K.

Embodiment 11. The composition of any of the preceding Embodiments, wherein the backward reaction activation energy (in kJ/mol) is less than a maximum thermal energy (in kJ) of the nanoparticle generated by absorption by the nanoparticle of a mole of photons at the NIR optical absorbance peak of the nanoparticle.

Embodiment 12. The composition of any of the preceding Embodiments, wherein the therapeutic species is an osteogenic modulator, a chondrogenic modulator, an endotheliologenic modulator, or a myogenic modulator.

Embodiment 13. The composition of Embodiment 12 for use in tissue regeneration.

Embodiment 14. The composition of any of the preceding Embodiments, wherein the therapeutic species is an anti-cancer agent.

Embodiment 15. The composition of Embodiment 14 for use in cancer chemotherapy.

Embodiment 16. The composition of Embodiment 12 or Embodiment 14, wherein the therapeutic species is a small molecule, a nucleic acid, a peptide, a protein, or any combination thereof.

Embodiment 17. The composition of Embodiment 16, wherein the nucleic acid comprises a plasmid, a small interfering RNA (“siRNA”), a micro-RNA (“miRNA”), a miRNA mimic, or any combination thereof.

Embodiment 18. The composition of any of the preceding Embodiments, wherein the therapeutic species comprises miRNA-34a-5p, miRNA-7-5p, miRNA-218-5p, miRNA-148b-3p, miRNA433-3p, miRNA-181a-5p, or a combination of two or more of the foregoing.

Embodiment 19. The composition of any of the preceding Embodiments, wherein the linker is covalently bonded to the nanoparticle.

Embodiment 20. The composition of any of the preceding Embodiments, wherein the linker is covalently bonded to the therapeutic species.

Embodiment 21. The composition of Embodiment 19 or Embodiment 20, wherein the linker is covalently bonded to the nanoparticle and/or to the therapeutic species through an R—S—X bond, where R is alkyl or another organic substituent bonded to sulfur through a carbon atom and X is the nanoparticle or the therapeutic species.

Embodiment 22. A method of delivering a therapeutic species to a biological compartment, the method comprising:
disposing the composition of any of Embodiments 1-21 in the biological compartment; and
initiating a retro Diels-Alder reaction to decompose the Diels-Alder cycle-addition product, thereby severing the linker and decoupling the therapeutic species from the nanoparticle.

Embodiment 23. A method of inducing tissue regeneration, the method comprising:
disposing the composition of any of Embodiments 1-21 in the biological compartment; and
initiating a retro Diels-Alder reaction to decompose the Diels-Alder cycle-addition product, thereby severing the linker and decoupling the therapeutic species from the nanoparticle,
wherein the therapeutic agent is a tissue regenerator.

Embodiment 24. A method of treating cancer, the method comprising:
disposing the composition of any of Embodiments 1-21 in the biological compartment; and
initiating a retro Diels-Alder reaction to decompose the Diels-Alder cycle-addition product, thereby severing the linker and decoupling the therapeutic species from the nanoparticle,
wherein the therapeutic agent is an anti-cancer agent.

Embodiment 25. The method of Embodiment 22 or Embodiment 23, wherein the therapeutic species is an osteogenic modulator, a chondrogenic modulator, an endotheliologenic modulator, or a myogenic modulator.

Embodiment 26. The method of Embodiment 22, wherein the therapeutic species is an anti-cancer agent.

Embodiment 27. The method of any of Embodiments 22-24, wherein the therapeutic species is a small molecule, or, a nucleic acid, a peptide, a protein, or any combination thereof.

Embodiment 28. The composition of Embodiment 27, wherein the nucleic acid comprises a plasmid, a small interfering RNA (“siRNA”), a micro-RNA (“miRNA”), a miRNA mimic, or any combination thereof.

Embodiment 29. The method of any of Embodiments 22-24, wherein initiating the retro Diels-Alder reaction comprises heating the nanoparticle to an activation temperature of the retro Diels-Alder reaction.

Embodiment 30. The method of Embodiment 29, wherein the activation temperature is between 45° C. and 150° C.

Embodiment 31. The method of Embodiment 29, wherein heating the nanoparticle to the activation temperature comprises exposing the nanoparticle to NIR light.

Embodiment 32. The method of Embodiment 31, wherein the NIR light has a wavelength distribution centered around a central exposure wavelength that is within 30 nm, within 20 nm, or within 10 nm of the NIR optical absorbance peak of the nanoparticle.

Embodiment 33. The method of Embodiment 32, wherein:
the nanoparticle absorbs at least a portion of the energy of the NIR light in a surface plasmon resonance process to provide an absorbed plasmon resonance energy;
the absorbed plasmon resonance energy is at least partially converted to thermal energy of the nanoparticle, thereby increasing a temperature of the nanoparticle; and
increasing the temperature of the nanoparticle results in heating the nanoparticle to the activation temperature of the Diels-Alder reaction.

Embodiment 34. The method of any of Embodiments 32 or 33, wherein the Diels-Alder cyclo-addition product does not absorb more than 5% of incident photons having a wavelength within the wavelength distribution of the NIR light.

Embodiment 35. The method of Embodiment 34, wherein the Diels-Alder cyclo-addition product does not absorb more than 5% of incident photons having a wavelength within 30 nm, within 20 nm, or within 10 nm of the NIR optical absorbance peak of the nanoparticle.

Various embodiments of the invention have been described in fulfillment of the various objectives of the invention. It should be recognized that these embodiments are merely illustrative of the principles of the invention. Numerous modifications and adaptations thereof will be readily apparent to those skilled in the art without departing from the spirit and scope of the invention.

1. A composition comprising:
a core-shell-shell nanoparticle comprising a core, a first shell overlying the core, and a second shell overlying the first shell;
a therapeutic species; and
a linker joining the nanoparticle to the therapeutic species,
wherein the linker comprises a Diels-Alder cyclo-addition reaction product,
wherein the nanoparticle has an optical absorbance peak in a near infrared (NIR) region of an electromagnetic spectrum, and
wherein the Diels-Alder cyclo-addition reaction product does not have an optical absorbance peak within 100 nm of the optical absorbance peak of the nanoparticle.

2. The composition of claim 1, wherein the nanoparticle comprises a gold-silver-gold core-shell-shell nanoparticle.

3. The composition of claim 1, wherein the second shell of the nanoparticle has an average thickness of at least 25 nm.

4. The composition of claim 1, wherein the nanoparticle has a surface roughness of no greater than 10 nm.

5. The composition of claim 1, wherein an exterior surface of the nanoparticle is free or substantially free of spikes or protrusions having a height of 2 nm or greater.

6. (canceled)

7. The composition of claim 1, wherein:
the nanoparticle comprises a gold-silver-gold core-shell-shell nanoparticle;
the nanoparticle has a diameter of 60 nm to 140 nm; and
the nanoparticle has a localized surface plasmon resonance (LSPR) peak within a range of 700 nm to 900 nm.

8. The composition of claim 1, wherein:
the Diels-Alder cyclo-addition reaction product has a forward reaction activation energy and a backward reaction activation energy, and
the backward reaction activation energy is at least 1.5 times the forward reaction activation energy.

9. The composition of claim 8, wherein the backward reaction activation energy is greater than a thermal energy provided by aqueous surroundings of the nanoparticle at a temperature of 310K.

10. The composition of claim 8, wherein the backward reaction activation energy (in kJ/mol) is less than a maximum thermal energy (in kJ) of the nanoparticle generated by absorption by the nanoparticle of a mole of photons at the NIR optical absorbance peak of the nanoparticle.

11. The composition of claim 1, wherein the therapeutic species is an osteogenic modulator, a chondrogenic modulator, an endotheliologenic modulator, a myogenic modulator, or an anti-cancer agent.

12-14. (canceled)

15. The composition of claim 1, wherein the therapeutic species is a small molecule, a nucleic acid, a peptide, a protein, or any combination thereof.

16. (canceled)

17. The composition of claim 1, wherein the therapeutic species comprises miRNA-34a-5p, miRNA-7-5p, miRNA-218-5p, miRNA-148b-3p, miRNA433-3p, miRNA-181a-5p, or a combination of two or more of the foregoing.

18. The composition of claim 1, wherein the linker is covalently bonded to the nanoparticle and/or the therapeutic species.

19-20. (canceled)

21. A method of delivering a therapeutic species to a biological compartment, the method comprising:
disposing the composition of claim 1 in the biological compartment; and
initiating a retro Diels-Alder reaction to decompose the Diels-Alder cyclo-addition reaction product, thereby severing the linker and decoupling the therapeutic species from the nanoparticle, wherein initiating the retro Diels-Alder reaction comprises heating the nanoparticle to an activation temperature of the retro Diels-Alder reaction.

22. A method of inducing tissue regeneration, the method comprising the method of claim 21,
wherein the therapeutic species is a tissue regenerator.

23. A method of treating cancer, the method comprising the method of claim 21,
wherein the therapeutic species is an anti-cancer agent.

24-28. (canceled)

29. The method of claim 21, wherein the activation temperature is between 45° C. and 150° C.

30. The method of claim 21, wherein heating the nanoparticle to the activation temperature comprises exposing the nanoparticle to NIR light, wherein the NIR light has a wavelength distribution centered around a central exposure wavelength that is within 30 nm, within 20 nm, or within 10 nm of the NIR optical absorbance peak of the nanoparticle.

31. (canceled)

32. The method of claim 30, wherein:
the nanoparticle absorbs at least a portion of the energy of the NIR light in a surface plasmon resonance process to provide an absorbed plasmon resonance energy;
the absorbed plasmon resonance energy is at least partially converted to thermal energy of the nanoparticle, thereby increasing a temperature of the nanoparticle; and
increasing the temperature of the nanoparticle results in heating the nanoparticle to the activation temperature of the Diels-Alder reaction.

33. The method of claim 32, wherein the Diels-Alder cyclo-addition product does not absorb more than 5% of incident photons having a wavelength within the wavelength distribution of the NIR light

34. (canceled)
